SEQUENCE LISTING (7 sequences)

SEQ ID NO: 1 - protein, 1039 aa, Homo sapiens
MAVQLVPDSALGLLMMTEGRRCQVHLLDDRKLELLVQPKLLAKELLDLVASHFNLKEKEYFGIAFTDETGHLNWLQLDRRVLEHDFPKKSGPVVLYFCVRFYIESISYLKDNATIELFFLNAKSCIYKELIDVDSEVVFELASYILQEAKGDFSSNEVVRSDLKKLPALPTQALKEHPSLAYCEDRVIEHYKKLNGQTRGQAIVNYMSIVESLPTYGVHYYAVKDKQGIPWWLGLSYKGIFQYDYHDKVKPRKIFQWRQLENLYFREKKFSVEVHDPRRASVTRRTFGHSGIAVHTWYACPALIKSIWAMAISQHQFYLDRKQSKSKIHAARSLSEIAIDLTETGTLKTSKLANMGSKGKIISGSSGSLLSSGSQESDSSQSAKKDMLAALKSRQEALEETLRQRLEELKKLCLREAELTGKLPVEYPLDPGEEPPIVRRRIGTAFKLDEQKILPKGEEAELERLEREFAIQSQITEAARRLASDPNVSKKLKKQRKTSYLNALKKLQEIENAINENRIKSGKKPTQRASLIIDDGNIASEDSSLSDALVLEDEDSQVTSTISPLHSPHKGLPPRPPSHNRPPPPQSLEGLRQMHYHRNDYDKSPIKPKMWSESSLDEPYEKVKKRSSHSHSSSHKRFPSTGSCAEAGGGSNSLQNSPIRGLPHWNSQSSMPSTPDLRVRSPHYVHSTRSVDISPTRLHSLALHFRHRSSSLESQGKLLGSENDTGSPDFYTPRTRSSNGSDPMDDCSSCTSHSSSEHYYPAQMNANYSTLAEDSPSKARQRQRQRQRAAGALGSASSGSMPNLAARGGAGGAGGAGGGVYLHSQSQPSSQYRIKEYPLYIEGGATPVVVRSLESDQEGHYSVKAQFKTSNSYTAGGLFKESWRGGGGDEGDTGRLTPSRSQILRTPSLGREGAHDKGAGRAAVSDELRQWYQRSTASHKEHSRLSHTSSTSSDSGSQYSTSSQSTFVAHSRVTRMPQMCKATSAALPQSQRSSTPSSEIGATPPSSPHHILTWQTGEATENSPILDGSESPPHQSTDE

SEQ ID NO: 2 - DNA, 6804 bp, Homo sapiens
gcagtgcaattccatgttcctcttaagtatgttagccctaccgggagctgagctggccagtctacttggagaggaaaagtagatctggggaaggtggaagggtcagttcctaagtgacttcctcctcggggatggtaagggcatttgctgatctccagtgactgcctggtgcctcatggtcagactcggctgtctcactcccagatatctgattttgcaaaaagggacacacctatctgcagcaaagaagacactgaccagattgcgagcggtgcttttggatgctctgtagccacccggggcccaggaggactgactcggcagcaggattcgtgcatgggaatcggagaccatggcagtgcagctggtgcccgactcagctctcggcctgctgatgatgacggagggccgccgatgtcaagtacatcttcttgatgacaggaagctggaactcctagtacagcccaagctgttggccaaggagcttcttgaccttgtggcttctcacttcaatctgaaggaaaaggagtactttggaatagcattcacagatgaaacgggacacttaaactggcttcagctagatcgaagagtattggaacatgacttccctaaaaagtcaggacccgtggttttatacttttgtgtcaggttctatatagaaagcatttcatacctgaaggataatgctaccattgagcttttctttctgaacgcgaagtcctgcatctacaaggagcttattgacgttgacagcgaagtggtgtttgaattagcttcctatattttacaggaggcaaagggagatttttctagcaatgaagttgtgaggagtgacttgaagaagctgccagcccttcccacccaagccctgaaggagcacccttccctggcctactgtgaagacagagtcattgagcactacaagaaactgaacggtcagacaagaggtcaagcaatcgtaaactacatgagcatcgtggagtctctcccaacctacggggttcactattatgcagtgaaggacaagcagggcataccatggtggctgggcctgagctacaaagggatcttccagtatgactaccatgataaagtgaagccaagaaagatattccaatggagacagttggaaaacctgtacttcagagaaaagaagttttccgtggaagttcatgacccacgcagggcttcagtgacaaggaggacgtttgggcacagcggcattgcagtgcacacgtggtatgcatgtccggcattgatcaagtccatctgggctatggccataagccaacaccagttctatctggacagaaagcagagtaagtccaaaatccatgcagcacgcagcctgagtgagatcgccatcgacctgaccgagacggggacgctgaagacctcgaagctggccaacatgggtagcaaggggaagatcatcagcggcagcagcggcagcctgctgtcttcaggttctcaggaatcagatagctcgcagtcggccaagaaggacatgctggctgccttgaagtccaggcaggaagctctggaggaaaccctgcgtcagaggctggaggaactgaagaagctgtgtctccgagaagctgagctcacgggcaagctgccagtagaatatcccctggatccaggggaggaaccacccattgttcggagaagaataggaacagccttcaaactggatgaacagaaaatcctgcccaaaggagaggaagctgagctggaacgcctggaacgagagtttgccattcagtcccagattacggaggccgcccgccgcctagccagtgaccccaacgtcagcaaaaaactgaagaaacaaaggaaaacctcgtatctgaatgcactgaagaaactgcaggagattgaaaatgcaatcaatgagaaccgcatcaagtctgggaagaaacccacccagagggcttcgctgatcatagacgatggaaacattgccagtgaagacagctccctctcagatgcccttgttcttgaggatgaagactctcaggttaccagcacaatatcccccctacattctcctcacaagggactccctcctcggccaccgtcgcacaacaggcctcctcctccccagtccctggagggactccgacagatgcactatcaccgcaacgactatgacaagtcacccatcaagcccaaaatgtggagtgagtcctctttagatgaaccctatgagaaggtcaagaagcgctcctctcacagccattccagcagccacaagcgcttccccagcacaggaagctgtgcggaagccggcggaggaagcaactccttgcagaacagccccatccgcggcctcccgcactggaactcccagtccagcatgccgtccacgccagacctgcgggtccggagtccccactacgtccattccacgaggtcggtggacatcagccccacccgactgcacagcctcgcactgcactttaggcaccggagctccagcctggagtcccagggcaagctcctgggctcggaaaacgacaccgggagccccgacttctacaccccgcggactcgtagcagcaacggctcagaccccatggacgactgctcgtcgtgcaccagccactcgagctcggagcactactacccggcgcagatgaacgccaactactccacgctggccgaggactcgccgtccaaggcgcgccagaggcagaggcagcggcagcgggcggcgggcgcactgggctcagccagctcgggcagcatgcccaacctggcggcgcgcgggggtgcggggggcgcggggggcgcggggggcggtgtgtacctgcacagccagagccagcccagctcgcagtaccgcatcaaggagtacccgctgtacatcgagggcggcgccacgcccgtggtggtgcgcagcctggagagcgaccaggagggccactacagcgtcaaggctcagttcaagacgtccaactcctacacggcgggcggcctgttcaaggagagctggcgcggcggcggcggcgacgagggcgacacgggccgcctgacgccgtcgcgatcgcagatcctgcggactccgtcgctgggccgcgagggcgcccacgacaagggcgcgggccgtgccgccgtctcagacgagctgcgccagtggtaccagcgttccaccgcctcgcacaaggagcacagccgcctgtcgcacaccagctccacctcctcggacagcggctcgcagtacagcacctcctcccagagcaccttcgtggcgcacagcagggtcaccaggatgccccagatgtgcaaggccacgtcagctgccttacctcaaagccagagaagctcgacaccgtcaagtgaaattggagccacccccccaagcagcccccaccacatcctaacctggcagactggagaagcaacagaaaactcacccattctggatgggtctgagtctccacctcaccaaagtactgatgaatagaggagctacaatgatagctgtttcctggattcctccctctatccagaactagctgatgtccagtggtacgggcaggaaaaagccaagcccgggaccctcgtgtgagccagcccggcctaatctgaccgcctcaacgccattctgagatcacctcactgcctctcatttgccttacccagacgcaccgtcaccctgcaccagctttggccctcagcactttttttctcctgtctccgcattccctcccccttgaaaacctgactgaggagacattctggaaggttccggtcccactgtgtgtcccctggcgctcttgcccatagagagccagacaccaatcctcaatggcaccttggtggcttccctctgccatgacagcccctaggccaggaaccatcaggggggccagccggcatccaattcctgcggataagtagcgttgggagagaacgggaaaggggacttgagttacagggtgacccagaaagacgattcagctgtgtccagcctgccacccatacgtaggccaaccaagcacttcatgaagaggaggcctcgtggcatattcagtttacacctgaaatattccttgatgggacagcttgtggggatggctatgggggaaggggaggttgagaaaggaagttctcgacaccagaaatgcatcggaggaccacaatcagttctatgctgccaaagattaaaaataaataaaaacataaaaaattaagaggggccaagaggaagacattctttctgcaaggaaatttcttttaaattctgaactgctactacacacaagtgaaagtcaaccctatgtaaactggtgtcctctctctagccctctcccttactggcccacttctctctccgtagagagcctgaaaaactgccccaatgccacggtaaaggcgaggaagtcttggctggcgttgctgactcacagtcgccatccatctggacacaaagagagacctgtgggagtcgtagagggtactgttagccccggtccatgcagggggttcagccgagcccaagactcaaagctgctttcctttcaggatttgtagtaacgtaaggtgataatggccaaaagtggttctctctcattaaaccaaccagtaaaagcgtatcctatttttttgcacaaggtgtttcattttcgtttttatgggaaaccaagggaaaagcacattgcgatccattcagtgtttaactgtcgtggctcattttctgttcgttagcacttgtgtgacaaaagagctcagatccgacttctcctatgtgtcacttattccaagaacccaactatgcccttaggtagaaagatttgactcgtgtgtctactagccaacaggcagagcagggttgaaaaaaatatcagctcccaaagggcccatgtgtctacatcatcagttactgtcatgcaccacatttgtgtgcagataccaaaagaggaggaaagaagaaaaaaattaatgtgtgggagctgcacgtttacatgttttgagctatgcttcaaacacaactggaaagccatcaatcttcaaaggcctcaaaaatacttttatagtaacaagtgcacgactttagttgggttattcaagatggcacaaaaaggtttccgcagaggtggtatgctgtgcttttggcgcaagtggtggggggatggggtgggggtggaatttttttctcactctaatgacttcctattggaaaggcattgacagccagggacaggagccagggtgggggtagttttgtgggaaagcagaactgaagttagcttaagcataaaaacaaagaaaaatcttcgcttttcatgtatgtggaatccaagaataaccataggctctaccagaccagaagggtaaggatggacactaaaatgaaacaaataccaaggtattccttctgctgcagcctggagaccaccgagagtcgagctggggcacacacacacctggccgggacccggcagggacaaggcgggccgtggcctcctccaccaagtctctctagacaattcagggcctgctttccccagctccatgcatggctggactggtgattccagggtgcagaagggattcatattcccagaacgctttaagtgtacacctgcaggataaagagataccggttacattattaaatgattctagggattcactgggggatatttttgttgcttttactttcatggttagagctacaaagaacagtgattttttttttttctcccttccccattcagaaacattatacattgggccatttttctttctcccaaagaagattcatggatagtcagactgaactgtgtgcaacaggaaaagtcaaaagggaaaaggtagctgatgaggttacatggttacatgttctacatcatgcatagtagcttgaaatctagtctggagaaaactggatcaagattctagcccactggagttgcaaggaatgagaggcaaaaattctaaagatttgggttatattttcaacttgggggacagagagaaatggagagcaggaattacagttccaacaaacatcatgatagtctggtagtcaagacagagattaagtaaaacaggttttactgtttagctgagttcagttaatacaaaatgtacataaaacgttagtcctttgagactgacatgattaatgatcagtgtggtgggaaatgatgtagttattgtacacaagcacttgcaaactctttatccctatttctttaaaacaaaataaggtgaaatacgaagtccttggtctgatataaagcccctattggattcttcggatgcgtaaaagaaattgcctgtttcagccagaagactggtgaaaacacatacatcagactatgttgtgagccaggttgattttttattttattatatgcaggtgagtgttgaaactgttaaaattccaatttgttttcattcagtattagtttagttctaaatatagcaaaccccatccaggtgctatcagatgaccagttactgcttagttaactaggtgtaaagttttacatatacattaatgtcaatagtttattacaagttgtgtaaaatggactctagtttaataatgggggaaaaaagattaggttgctcctgaaactgactgtagagcatgtaaaatgattttactggattctgttcaactgtaatcaatgaaaaagatgtacgttgtagacaaagttgcagaattaaaaaaagaaatctgcttttaatttattctttttgtattaagaatttgtatagtacctttacattttgcaaaacagtgttgtcaacacttattaaagcattttcaaaatgaaaaga

SEQ ID NO: 3 - protein, 21 aa, Homo sapiens
IAFTDETGHLNWLQLDRRVLE

SEQ ID NO: 4 - protein, 21 aa, Homo sapiens
PHKGLPPRPPSHNRPPPPQSL

SEQ ID NO: 5 - protein, 21 aa, Homo sapiens
ATENSPILDGSESPPHQSTDE

SEQ ID NO: 6 - protein, 21 aa, Homo sapiens
DRRVLEHDFPKKSGPVVLYFC

SEQ ID NO: 7 - protein, 303 aa, Homo sapiens
RRCQVHLLDDRKLELLVQPKLLAKELLDLVASHFNLKEKEYFGIAFTDETGHLNWLQLDRRVLEHDFPKKSGPVVLYFCVRFYIESISYLKDNATIELFFLNAKSCIYKELIDVDSEVVFELASYILQEAKGDFSSNEVVRSDLKKLPALPTQALKEHPSLAYCEDRVIEHYKKLNGQTRGQAIVNYMSIVESLPTYGVHYYAVKDKQGIPWWLGLSYKGIFQYDYHDKVKPRKIFQWRQLENLYFREKKFSVEVHDPRRASVTRRTFGHSGIAVHTWYACPALIKSIWAMAISQHQFYLDRK

(19) United States
(12) Patent Application Publication
(10) Pub. No.: US 2014/0023589 A1
(43) Pub. Date: Jan. 23, 2014

(54) FRMD4A ANTAGONISTS AND THEIR USES

(71) Applicants: Fiona Watt, London (GB); Stephen Goldie, London (GB)

(72) Inventors: Fiona Watt, London (GB); Stephen Goldie, London (GB)

(73) Assignee: CANCER RESEARCH TECHNOLOGY LIMITED, London (GB)

(21) Appl. No.: 13/984,520

(22) PCT Filed: Feb. 9, 2012
(86) PCT No.: PCT/GB2012/000137

§ 371 Date: Oct. 9, 2013

(30) Foreign Application Priority Data
Feb. 9, 2011 (US) 61440995

(51) Int. Cl. A61K 39/395

(52) U.S. Cl. CPC A61K 39/39558
USPC 424/9.2; 424/139.1; 424/133.1; 530/387.9; 424/135.1; 424/136.1; 435/7.1

(57) ABSTRACT

An antagonist of FERM domain-containing protein 4A (FRMD4A) and/or of the Hippo pathway for use in a method of treating a cancer in a mammalian subject, wherein the cancer is selected from: squamous cell carcinoma (SCC), an epithelial cancer, an adenocarcinoma and a carcinoma is disclosed, as well as related methods of treatment of cancer, methods of screening and generating such antagonists, including anti-FRMD4A antibodies.

### FIELD OF THE INVENTION

The present invention relates to agents that inhibit FERM domain-containing protein 4A (“FRMD4A”) and/or the Hippo pathway, including anti-FRMD4A antibodies, and to methods of producing, screening and using such agents, such as in therapeutic methods for treating proliferative disorders, including squamous cell carcinoma (SCC).

### BACKGROUND TO THE INVENTION

Squamous cell carcinoma (SCC) arising in the skin and oral cavity represent varying problems to the health of an individual. SCC tumours can develop from many different tissue types including the skin, lips, mouth, oesophagus, bladder, prostate, lungs, vagina and cervix. Tumours developing in the skin tend to be less aggressive, than the oral cavity, but may still metastasise. Local control of cutaneous lesions may involve extensive, disfiguring surgery. SCCs in the oral cavity tend to develop insidiously and present at an advanced stage. Despite radical surgery and adjuvant therapy, these lesions often recur and spread to other body sites. Survival rates for oral SCC have not improved for 30 years. Due to the rapid turnover of the stratified squamous epithelium in skin and the oral cavity, presumably only the long-term residents of the stem cell niche are present for sufficient time to accumulate the genetic changes required to form a malignancy. In SCCs there is increased proliferation, a reduced proportion of the cells undergo terminal differentiation, and the spatial organisation of the tissue is disrupted.

Recent interest has focused on the existence of tumour stem cells, that is, cells that may constitute a minority of the cells in a tumour, but are responsible for tumour re-growth following conventional treatment (Reya et al., 2001). Treatments that are specific for cancer stem cells will not only be more effective in treating the disease but may also be less damaging to the patient's normal tissues. In the case of SCCs of the head and neck (HNSCC) there is evidence from clonal growth analysis of human SCC lines (Locke et al., 2005) and xenografts of SCC cell subpopulations in immune compromised mice (Prince et al., 2007) for the existence of tumour stem cells. FRMD4A was identified as a potential marker of the stem cell compartment in human interfollicular epidermis (IFE), (Jensen and Watt, 2006). Further work showed that FRMD4A was overexpressed in a panel of SCC lines, and therefore a potential marker for cancer stem cells (Jensen et al., 2008). FERM domain-containing proteins similar to FRMD4A may regulate upstream events in the Hippo pathway, which has been implicated in the dysregulation of growth that is seen in the development cancer.

There remains a need to provide new therapeutic approaches to the treatment of SCCs. The present invention addresses this and other needs.

### SUMMARY OF THE INVENTION

Broadly, the present invention provides products and methods for modulating FRMD4A and/or the Hippo pathway and which find use in the management and treatment of certain proliferative disorders, particularly SCCs. The present inventors found that knock-down of FRMD4A expression, targeting FRMD4A using antibodies and manipulating the Hippo pathway using compounds that target the receptor, CD44, or the heat shock protein, HSP90, are approaches that reduce the growth of SCC cells in vitro and in vivo. Moreover, as described herein, antagonising FRMD4A was found to: increase animal survival in vivo; reduce SCC cell proliferation and increase apoptosis; reduce invasion and metastasis; and increase cellular differentiation of SCC cells. Therefore, the results described herein represent evidence that FRMD4A is not merely a marker of certain cancer stem cells, but is a functional protein in such cells and an attractive therapeutic target both for cancer stem cells as well as cancer cells more generally. The ability of an antibody directed to FRMD4A to exert an anti-cancer effect in vitro and in vivo is surprising not least because of the previously presumed intra-cellular location of FRMD4A. Previous reports have described a role for FRMD4A as a cytoskeletal intracellular protein (Ikenouchi and Umeda, 2010, Proc. Natl. Acad. Sci. USA, 107(2): 748-753).

Accordingly, in a first aspect the present invention provides an antagonist of FERM domain-containing protein 4A (FRMD4A) and/or of the Hippo pathway for use in a method of treating a cancer in a mammalian subject. Preferably, the cancer is selected from: squamous cell carcinoma (SCC), an epithelial cancer, an adenocarcinoma and a carcinoma.

In some cases in accordance with this and other aspects of the present invention, the antagonist may be selected from: an antibody molecule that specifically binds to FRMD4A, a nucleic acid molecule that inhibits expression of FRMD4A, an aptamer that specifically binds to FRMD4A, an affinity protein that specifically binds to FRMD4A, hyaluronic acid and 17-Dimethylaminoethylamino-17-demethoxygeldanamycin (17-DMAG).

In certain cases in accordance with this and other aspects of the present invention, the FRMD4A may have at least 90%, at least 95%, at least 99% or 100% amino acid sequence identity with the full-length human FRMD4A protein, the amino acid sequence of which is set forth in SEQ ID NO: 1. In certain cases, the FRMD4A may comprise the amino acid sequence of SEQ ID NO: 1, wherein 1, 2, 3, 4, 5, 10, 20 or 50 amino acids have been altered by substitution, insertion or deletion.

In certain cases in accordance with this and other aspects of the present invention, the antagonist may be an antibody molecule that specifically binds to an FRMD4A polypeptide or to a peptide fragment thereof. The FRMD4A polypeptide to which the antibody molecule specifically binds may have at least 90%, at least 95%, at least 99% or 100% amino acid sequence identity with the full-length human FRMD4A protein, the amino acid sequence of which is set forth in SEQ ID NO: 1. In certain cases, the FRMD4A polypeptide to which the antibody molecule binds may comprise the amino acid sequence of SEQ ID NO: 1, wherein 1, 2, 3, 4, 5, 10, 20 or 50 amino acids have been altered by substitution, insertion or deletion.

In certain cases in accordance with this and other aspects of the present invention, the antagonist may be an antibody molecule that specifically binds to a fragment of a FRMD4A polypeptide as defined herein. The fragment of the FRMD4A polypeptide may be a peptide consisting of a sequence of 5, 6, 7, 8, 9, 10, 11, 12, 15, 20, 50, 100, 200, 300, 400 or 500 contiguous amino acids of a FRMD4A polypeptide as defined herein, in particular, of the human FRMD4A amino acid sequence as set forth in SEQ ID NO: 1.

In certain cases in accordance with this and other aspects of the present invention, the antibody molecule may bind to FRMD4A or a fragment thereof in a region that corresponds to the FERM domain of FRMD4A. For example, the antibody molecule may bind to FRMD4A in a region corresponding to or defined by the contiguous sequence of residues 20-322 of the amino acid sequence set forth in SEQ ID NO: 1 (SEQ ID NO: 7). In certain preferred cases in accordance with this and other aspects of the present invention, the antibody molecule may bind to FRMD4A or a fragment thereof in the region defined by residues 63-83 (SEQ ID NO: 3), or 1019-1039 (SEQ ID NO: 5), or 78-98 (SEQ ID NO: 6) of the sequence of SEQ ID NO: 1.

In certain cases in accordance with this and other aspects of the present invention the antibody molecule may be an anti-FRMD4A antibody molecule that tests positive as an inhibitor of SCC growth, including growth rate, proliferation and/or cell number. This functional property of the antibody molecule may be assessed using any suitable assay, whether in vitro or in vivo. The antibody molecule may cause at least 10%, at least 20%, at least 30%, at least 40% or at least 50% reduction in the growth rate, proliferation and/or cell number of cultured SCC cells as compared with cultured SCC cells grown under identical conditions, but in the absence of said antibody molecule. The cultured SCC cells may comprise primary SCC culture and/or an established SCC cell line (e.g. SCC13, SCC25 or SJG-15). The reduction in growth, proliferation and/or cell number may be assessed over any suitable period, e.g. 24, 48, 96, 120 or more hours.

In certain cases in accordance with this and other aspects of the present invention the antibody molecule may be selected from: a polyclonal antibody, a monoclonal antibody, an intrabody, a complete antibody, a single domain antibody, a nanobody, a Fab fragment, a F(ab′)2 fragment, a scFv, a diabody, a triabody, a human antibody, a humanised antibody, a bispecific antibody and a chimeric antibody. For example, the antibody molecule of the present invention may comprise a monoclonal antibody generated using HuCAL technology (AbD Serotec), in particular, using HuCAL technology directed against the peptide consisting of the amino acid sequence set forth in SEQ ID NO: 6.

In certain cases in accordance with this and other aspects of the present invention the antagonist comprises a nucleic acid molecule that inhibits expression of FRMD4A. The antagonist comprising a nucleic acid may, for example, be selected from: shRNA, siRNA, miRNA, antisense RNA, antisense DNA and a ribozyme. The nucleic acid may be provided in the form of an artificial construct, for example the nucleic acid antagonist may be in the form of a viral construct, e.g. a lentiviral construct. The viral construct may facilitate delivery of the antagonist nucleic acid to a cell and/or may facilitate effective knock-down of FRMD4A expression by the cell. In some cases, the nucleic acid molecule may comprise shRNA. In some cases provided in the form of a shRNA lentiviral construct. The nucleic acid antagonist may inhibit expression of an FRMD4A polypeptide, as defined herein, by virtue of a direct or indirect effect on any step in the process of gene expression, including for example by inhibiting translation, transcription or decreasing mRNA stability. The nucleic acid antagonist may inhibit expression of FRMD4A, at least partly, by interacting with an mRNA having at least 90%, at least 95% or at least 99% or 100% nucleotide sequence identity to the full length sequence disclosed at NCBI accession number NM_{—}018027; GI: 116063561 (SEQ ID NO: 2). The nucleic acid antagonist may be produced using standard techniques directed against a gene or RNA that encodes a FRMD4A polypeptide as defined herein, e.g. directed against the mRNA having the sequence disclosed at NCBI accession number NM_{—}018027; GI: 116063561 (SEQ ID NO: 2). Additionally or alternatively, the nucleic acid antagonist may be obtained from commercial sources, e.g. shRNA against human FRMD4A is available from Open Biosystems.

In certain cases in accordance with this and other aspects of the present invention the antagonist that comprises a nucleic acid molecule may be an antagonist that tests positive as an inhibitor of SCC growth, including growth rate, proliferation and/or cell number. This functional property of the nucleic acid molecule may be assessed using any suitable assay, whether in vitro or in vivo. The nucleic acid antagonist may cause at least 10%, at least 20%, at least 30%, at least 40% or at least 50% reduction in the growth rate, proliferation and/or cell number of cultured SCC cells as compared with cultured SCC cells grown under identical conditions, but in the absence of said nucleic acid. The cultured SCC cells may comprise primary SCC culture and/or an established SCC cell line (e.g. SCC13, SCC25 or SJG-15). The reduction in growth proliferation and/or cell number may be assessed over any suitable period, e.g. 24, 48, 96, 120 or more hours.

In a second aspect the present invention provides a method of treating a mammalian subject having a cancer, the method comprising administering a therapeutically effective amount of an antagonist in accordance with the first aspect of the invention to the subject. Preferably, the cancer is selected from: squamous cell carcinoma (SCC), an epithelial cancer, an adenocarcinoma and a carcinoma.

In a third aspect the present invention provides use of an antagonist as defined in accordance with the first aspect of the invention in the preparation of a medicament for use in a method of treating a cancer in a mammalian subject. Preferably, the cancer is selected from squamous cell carcinoma (SCC), an epithelial cancer, an adenocarcinoma and a carcinoma.

In accordance with the first, second, third or any other aspect of the present invention, the mammalian subject is preferably a human. The subject may have been diagnosed as having or being susceptible to developing a cancer, including an SCC. In some cases the subject may have had surgical and/or pharmaceutical treatment for a cancer, including an SCC (e.g. the treatment in accordance with the present invention may be of a subject who or that has had surgical resection of an SCC tumour).

In accordance with the first, second, third or any other aspect of the present invention, the cancer may comprise a cancer of a tissue or organ selected from: skin, oral cavity, tongue, head, neck, lips, mouth, oesophagus, urinary bladder, prostate, lung, vagina, cervix, kidney, thyroid, mammary papilla, breast, liver and colon. In certain cases, the cancer is SCC of the head and neck (HNSCC).

In accordance with the first, second, third or any other aspect of the present invention the method of treating the cancer may comprises:
(i) a decrease in the rate of growth;
(ii) an increase in apoptosis;
(iii) an increase in the cellular differentiation;
(iv) a decrease in metastasis; and/or
(v) a decrease in the invasion,
   of one or more cancer cells. Additionally or alternatively the method of treating may comprise reducing the number of cancer stem cells in a tumour, such as in an SCC tumour. Reducing the number of cancer stem cells may comprise, for example, direct cell killing or inducing changes in the cancer stem cells towards non-stem cell phenotype (such as inducing differentiation of cancer stem cells).

In a fourth aspect the present invention provides an antagonist of FERM domain-containing protein 4A (FRMD4A), wherein the antagonist is selected from: an antibody molecule that specifically binds to FRMD4A or a fragment thereof, an aptamer that specifically binds to FRMD4A or a fragment thereof and an affinity protein that specifically binds to FRMD4A or a fragment thereof.

In some cases in accordance with the fourth aspect of the invention the anatagonist is an antibody molecule as defined in accordance with the first aspect of the invention. The antibody molecule may specifically bind to an FRMD4A polypeptide or to a peptide fragment thereof. The FRMD4A polypeptide to which the antibody molecule specifically binds may have at least 90%, at least 95%, at least 99% or 100% amino acid sequence identity with the full-length human FRMD4A protein, the amino acid sequence of which is set forth in SEQ ID NO: 1. In certain cases, the FRMD4A polypeptide to which the antibody molecule binds may comprise the amino acid sequence of SEQ ID NO: 1, wherein 1, 2, 3, 4, 5, 10, 20 or 50 amino acids have been altered by substituion, insertion or deletion.

In certain cases in accordance with this and other aspects of the present invention, the antibody molecule specifically binds to a fragment of a FRMD4A polypeptide as defined herein.

The fragment of the FRMD4A polypeptide may be a peptide consisting of a sequence of 5, 6, 7, 8, 9, 10, 11, 12, 15, 20, 50, 100, 200, 300, 400 or 500 contiguous amino acids of a FRMD4A polypeptide as defined herein, in particular, of the human FRMD4A amino acid sequence as set forth in SEQ ID NO: 1.

In certain cases in accordance with this and other aspects of the present invention, the antibody molecule may bind to FRMD4A or a fragment thereof in a region that corresponds to the FERM domain of FRMD4A. For example, the antibody molecule may bind to FRMD4A in a region corresponding to or defined by the contiguous sequence of residues 20-322 of the amino acid sequence set forth in SEQ ID NO: 1 (SEQ ID NO: 7). In certain preferred cases in accordance with this and other aspects of the present invention, the antibody molecule may bind to FRMD4A or a fragment thereof in the region defined by residues 63-83 (SEQ ID NO: 3) or 1019-1039 (SEQ ID NO: 5) or 78-98 (SEQ ID NO: 6) of the sequence of SEQ ID NO: 1.

In a fifth aspect the present invention provides an antagonist of FERM domain-containing protein 4A (FRMD4A) for use in medicine, wherein the antagonist is selected from: an antibody molecule that specifically binds to FRMD4A or a fragment thereof, a nucleic acid that inhibits the expression of FRMD4A, an aptamer that specifically binds to FRMD4A or a fragment thereof and an affinity protein that specifically binds to FRMD4A or a fragment thereof. The antagonist of the fifth aspect of the invention may be as defined in accordance with the fourth aspect of the invention.

In a sixth aspect the present invention provides an antagonist in accordance with the fifth aspect of the invention for use in a method of treating cancer in a mammalian subject.

In a seventh aspect the present invention provides a method of treating cancer in a mammalian subject, the method comprising administering a therapeutically effective amount of an antagonist in accordance with the fifth aspect of the invention to the subject.

In an eighth aspect the present invention provides use of an anatagonist in accordance with the fifth aspect of the invention in the preparation of a medicament for use in a method of treating cancer in a mammalian subject.

In some cases in accordance with the sixth, seventh, eighth of other aspects of the invention, the cancer may be an epithelial cancer. In particular, the cancer may be a cancer of a tissue or organ selected from: skin, oral cavity, tongue, head, neck, lips, mouth, oesophagus, urinary bladder, prostate, lung, vagina, cervix, kidney, thyroid, mammary papilla, breast, liver and colon. In some cases in accordance with the sixth, seventh, eighth of other aspects of the invention, the antagonist may be an antibody molecule as defined in accordance with the first aspect of the invention. In some cases in accordance with the sixth, seventh, eighth of other aspects of the invention, the subject is preferably a human. The subject may have been diagnosed as having or being susceptible to developing a cancer, such as an epithelial cancer. In some cases the subject may have had surgical and/or pharmaceutical treatment for a cancer, such as an epithelial cancer.

In a ninth aspect the present invention provides a pharmaceutical composition comprising an antagonist in accordance with the fourth or fifth aspect of the invention and a pharmaceutically acceptable carrier or excipient.

In a tenth aspect the present invention provides an isolated peptide consisting of:
(i) the contiguous sequence of residues 63 to 83 of the amino acid sequence set forth in SEQ ID NO: 1 (SEQ ID NO: 3); or
(ii) the contiguous sequence of residues 1019 to 1039 of the amino acid sequence set forth in SEQ ID NO: 1 (SEQ ID NO: 5); or
(iii) the contiguous sequence of residues 78 to 98 of the amino acid sequence set forth in SEQ ID NO: 1 (SEQ ID NO: 6).

In an eleventh aspect the present invention provides use of an isolated peptide as defined as defined in accordance with the tenth aspect for the production of an antibody molecule that specifically binds to FRMD4A, an aptamer that specifically binds to FRMD4A or an affinity protein that specifically binds to FRMD4A.

In a twelfth aspect the present invention provides a method of screening antibody molecules that specifically bind to FERM domain-containing protein 4A (FRMD4A). The method may be an in vitro, the method comprising:
(i) providing a plurality of antibodies directed to a FRMD4A polypeptide and/or one or more peptide fragments of said FRMD4A polypeptide; and
(ii) screening said antibodies from (i) for the ability to decrease the growth of cultured SCC cells in an in vitro cell growth assay as compared with the growth of cultured SCC cells grown under identical conditions, but in the absence of any of said antibodies; and
optionally (iii) isolating the or those antibodies that screen positive for said ability in (ii); and
optionally (iv) determining the sequence of at least the complementarity determining regions (CDRs) of one or more of said antibodies isolated in (iii).

Additionally or alternatively the method of the twelfth aspect of the invention may be an in vivo method of screening antibody molecules that specifically bind to FERM domain-containing protein 4A (FRMD4A), the method comprising:
(i) providing a plurality of antibodies directed to a FRMD4A polypeptide and/or one or more peptide fragments of said FRMD4A polypeptide; and
(ii) screening said antibodies from (i) for the ability to decrease the growth of an SCC tumour in vivo as compared with the growth of an SCC tumour in vivo that has not been treated with any of said antibodies; and
optionally (iii) isolating the or those antibodies that screen positive for said ability in (ii); and
optionally (iv) determining the sequence of at least the complementarity determining regions (CDRs) of one or more of said antibodies isolated in (iii).

In some cases in accordance with the twelfth aspect of the invention, the antibody molecule may be as defined in accordance with the first aspect of the invention.

In a thirteenth aspect the present invention provides a method of inhibiting growth of a tumour, the method comprising contacting at least one cancer stem cell of the tumour with an antagonist as defined in accordance with the first aspect of the invention.

The method of the thirteenth aspect of the invention may be carried out in vitro or in vivo.

In some cases in accordance with the method of the thirteenth aspect of the invention, the at least one cancer stem deli expresses FRMD4A. In some cases, the cancer is selected from: squamous cell carcinoma (SCC), an epithelial cancer, an adenocarcinoma and a carcinoma. The cancer may be as defined in accordance with the first aspect of the invention.

Embodiments of the present invention will now be described by way of example and not limitation with reference to the accompanying figures. However various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

The present invention includes the combination of the aspects and preferred features described except where such a combination is clearly impermissible or is stated to be expressly avoided. Section headings are used herein are for convenience only and are not to be construed as limiting in any way.

Unless context dictates otherwise, the descriptions and definitions of the features set out herein are not limited to any particular aspect or embodiment of the invention and apply equally to all aspects and embodiments which are described.

### BRIEF DESCRIPTION OF THE FIGURES

FIG. 1 shows A) in-situ hybridisation showing that FRMD4A is localised to the basal layer of skin tissue. Beta-actin is a control which also highlights the basal layer. B) shows that the known marker of differentiation TG1 is present in granular cells but not in the basal layer. FRMD4A has the opposite pattern i.e. it is expressed in the less differentiated basal layer. C) shows that induction of differentiation in keratinocytes with agents such as AG1478 and BMP2/7 leads to upregulation of TG1 levels and downregulation of FRMD4A levels. D) shows a schematic representation of the FRMD4A protein. SGO-1 and -2 polyclonal antibodies were generated against a peptide corresponding to residues 63-83 of the protein. SGO-3 and -4 polyclonal antibodies were generated against a peptide from 568-588 of the protein and SGO-5 and -6 were generated against a peptide of residues 1019-1039 of the protein.

FIG. 2 shows A) specificity of the FRMD4A polyclonal antibodies. SGO-1 detects protein in wild type SCC13 cells and the scrambled control line, but does not detect protein in the FRMD4A shRNA knockdown cell line (SCC13-A7). SGO-3 detects protein in wild type and scrambled control cells but not in the FRMD4A shRNA knockdown cell line (SCC13-A7). B) Shows staining of FRMD4A in basal layer of normal skin (upper panel) and widespread expression in SCC tumour tissue (lower panel).

FIG. 3 shows A) SCC cell lines (wild type (WT) versus scrambled shRNA control (SCR) versus FRMD4A shRNA knockdown SCC13-A7)). Left panels, cells stained with E-cadherin (cell surface adhesion molecule) in red. Right hand panels stained with Alpha-6 integrin (Red). FRMD4A staining (green) is seen mainly in cytoplasm but also at cell borders. Much of the e-cadherin cell border staining is lost following knock down of FRMD4A. B) Shows that shRNA knockdown of FRMD4A (SCC13-A7 line) reduces colony formation compared to wild type or scrambled shRNA control SCC13 cell lines. C) shRNA Knockdown of FRMD4A also leads to a reduction in the growth rate of SCC13-A7 cells compared to wild type or scrambled shRNA control SCC13 cell lines. SCC13 line is derived from a facial epidermis Squamous cell cancer. D) shRNA Knockdown of FRMD4A also reduces colony formation of the oral cavity derived SCC25 cell line (SCC25-A7). E) shRNA Knockdown of FRMD4A also inhibits the growth rate of the SCC25 cell line (SCC25-A7) compared to the wild type (WT) and scrambled (SCR) control SCC25 cell lines. SCC25 is derived from an oral cavity squamous cell carcinoma.

FIG. 4 shows a schematic representation of the in vivo experiments.

FIG. 5 shows growth of xenografts generated from SCC cell lines. A) Stable knockdown of FRMD4A (SCC25-A7 line) reduces the growth rate of these cells as xenografts compared to wild type or scrambled shRNA control SCC25 cell lines. This is translated into a significant improvement in the survival of these mice. B) Shows results from an inducible shRNA system. Xenografts from a SCC13 cell line containing an inducible shRNA against FRMD4A were allowed to become established for approximately 3 weeks and then expression of the FRMD4A shRNA was induced by adding tetracycline. FRMD4A shRNA mediated knock down reduces the tumour growth rate. This translates into a reduction in the tumour size following FRMD4A shRNA knock down and leads to improved survival. C) Immunohistochemical analysis of tumours from FIG. 5B show that FRMD4A shRNA knockdown leads to a reduced number of proliferating cells per area of tumour and a reduction in the percentage of proliferating cells in the tumour as assessed by cellular staining for the proliferation marker Ki67. Immunohistochemical staining also demonstrates an increase in staining for the apoptosis marker cleaved caspase-3 in cells with FRMD4A knocked down by shRNA.

FIG. 6 shows A) using induction of FRMD4A shRNA in xenografts demonstrated that knockdown of FRMD4A results in significantly less metastases of the SCC13 cells into both the lung and the liver as compared to the levels seen in the empty vector control (EV). B) Boyden chamber assay showing that shRNA knockdown of FRMD4A leads to a reduced migration of the SCC13 cells. This was then confirmed in a matrigel invasion assay where loss of FRMD4A (SCC13-A7 line) greatly reduced invasion as compared to the wild type (WT) and scrambled shRNA control (SCR) SCC13 cell lines. C) The reduction in the migratory/invasive and metastatic phenotype correlates with a reduction in the level of proteins associated with metastasis (vimentin and snail) in SCC13 cells where FRMD4A has been knocked down as compared to the empty vector (EV) control cells. D) Injection into the bloodstream of SCC13 cells with FRMD4A knocked down (SCC13-A7) results in a reduction in the number of cells subsequently found to have lodged in the lungs of mice (measure of metastatic ability) as compared to the wild type (WT) and scrambled shRNA control (SCR) SCC13 cell lines.

FIG. 7 shows A) shRNA knockdown of FRMD4A in SCC xenografts induces differentiation of the cells and this correlates with the appearance of the differentiation marker (involucrin) in SCC cells after FRMD4A knock down as compared to the empty vector (EV) control SCC xenografts. B) Role for FRMD4A in the Hippo pathway. Knockdown of FRMD4A (SCC13-A7) leads to an increase in the levels of the Hippo pathway transcription factor YAP (bottom panel western blot) compared to the wild type (WT) and scrambled (SCR) shRNA control cells. Knock down of FRMD4A also leads to a change in the distribution of YAP with more locating to the nucleus (upper panel). C) A similar change to YAP localisation (to the nucleus) is also seen in SCC xenografts where FRMD4A has been knocked down and a reduction in the amount of LATS1 (another Hippo pathway component) was seen compared to the empty vector (EV) control cells.

FIG. 8 shows A) that on a plot of cellular confluence (y-axis) versus time α-axis) treatment of an SCC cell line (SCC13) with a polyclonal antibody against FRMD4A (SGO-1)(triangles) reduces the growth rate of the cells as compared to the non-inhibitory control antibody SGO-4. B) Shows that on a plot of cellular confluence (y-axis) versus time (x-axis) treatment of another SCC cell line (SCC25) with a polyclonal antibody against FRMD4A (SGO-1)(triangles) reduces the growth rate of the cells as compared to the non-inhibitory control antibody SGO-4. C) Shows the effect of FRMD4A-targeting polyclonal antibodies SGO-1, -2, -3, -4, -5 and -6 on cell growth of the SCC cell line SCC13. SGO-1, SGO-6, SGO-2, and SGO-5 exhibited the greatest reduction in SCC cell growth. D) Shows the effect of FRMD4A-targeting polyclonal antibodies SGO-1, -2, -3, -4, -5 and -6 on cell growth of the SCC cell line SCC25. SGO-1, SGO-6 and SGO-5 exhibited the greatest reduction in SCC cell growth. E) Treatment of SCC25 tongue orthotopic xenograft with a single intraperitoneal dose of the FRMD4A polyclonal antibody (SGO-1) leads to significant reduction in the growth of these tumours. Treatment with the SGO-4 polyclonal raised against a different region of FRMD4A (this polyclonal does not have anti-proliferative activity against SCC cell lines) has no effect on growth of the SCC xenografts. WIB10 is an IgG control antibody which also has no effect on tumour growth. F) Treatment of SCC25 tongue xenograft mice with a single intraperitoneal dose of the FRMD4A polyclonal antibody (SGO-1) also leads to significant reduction in the metastatic disease burden of the mice compared to the WIB10 IgG control treated mice.

FIG. 9 A)-F) tissue microarray showing FRMD4A staining of normal tissue versus tumour tissue from a range of cancer types. FRMD4A is present on a number of epithelial cancers including prostate and breast cancers in addition to SCCs.

FIG. 10 shows Western Blots of SCC13 cells treated with siRNA targeting human FRMD4A (A7) or scramble control siRNA (SCR) and levels of FRMD4A determined using 5 distinct monoclonal antibodies, (GOLDIE-1, GOLDIE-2, GOLDIE-3, GOLDIE-4 and GOLDIE-5). GAPDH was used to confirm equal protein loading.

FIG. 11 shows percentage confluence of A) SCC13 cells and B) SCC25 cells treated with either scramble control siRNA (Scr) or FRMD4A siRNA (A7) in the presence of control IgG (triangles) or the SGO-1 anti-FRMD4A antibody (diamonds).

References to colour in the figures are for guidance only; the figures do not contain colour.

### DETAILED DESCRIPTION

### Antagonists of FRMD4A

The present invention provides antagonists of an FRMD4A polypeptide or to a peptide fragment thereof, and uses of such antibody molecules such as in therapeutic methods of treating mammalian subjects having cancer, particularly SCC or (other) epithelial cancer (including carcinomas and adenocarcinomas).

A number of formats of FRMD4A antagonists are contemplated in accordance with the present invention. Preferably, the antagonist comprises an antibody molecule as defined herein. However, nucleic acid-based antagonists that inhibit expression of FRMD4A find use in the methods of the invention.

The FRMD4A antagonist of the invention may comprise an aptamer (including an oligonucleotide aptamer or a peptide aptamer). Advantageously, an aptamer directed to FRMD4A may be provided using a technique such as that known as SELEX (Systematic Evolution of Ligands by Exponential Enrichment), described in U.S. Pat. Nos. 5,475,096 and 5,270,163.

Non-immunoglobulin-based specific binding proteins can also be engineered against target proteins to generate functional blocking agents. Therefore, the FRMD4A antagonist of the invention may comprise a non-antibody-based specific binding protein. An example specifically contemplated in accordance with the present invention is a lipocalin-based anticalin that specifically binds an FRMD4A polypeptide or fragment thereof as defined herein.

The FRMD4A anatagonist of the invention may comprise an affinity protein such as an affinity protein described in Friedman and Stahl, 2009, Biotechnol. Appl. Biochem., 53, 1-29, the contents of which are incorporated herein by reference.

The FRMD4A anatagonist of the invention may comprise an engineered protein scaffold as described in Gebauer and Skerra, 2009, Current Opinion in Chemical Biology, 13, 245-255, the contents of which are incorporated herein by reference.

### Anti-FRMD4A Antibody Molecules

The present invention provides antibody molecules that specifically bind to an FRMD4A polypeptide or to a peptide fragment thereof, and uses of such antibody molecules such as in therapeutic methods of treating mammalian subjects having cancer, particularly SCC or (other) epithelial cancer. The FRMD4A polypeptide to which the antibody molecule specifically binds may have at least 90%, at least 95%, at least 99% or 100% amino acid sequence identity with the full-length human FRMD4A protein, the amino acid sequence of which is set forth in SEQ ID NO: 1. In certain cases, the FRMD4A polypeptide to which the antibody molecule binds may comprise the amino acid sequence of SEQ ID NO: 1, wherein 1, 2, 3, 4, 5, 10, 20 or 50 amino acids have been altered by substituion, insertion or deletion.

In certain cases in accordance with this and other aspects of the present invention, the antagonist may be an antibody molecule that specifically binds to a fragment of a FRMD4A polypeptide as defined herein. The fragment of the FRMD4A polypeptide may be a peptide consisting of a sequence of 5, 6, 7, 8, 9, 10, 11, 12, 15, 20, 50, 100, 200, 300, 400 or 500 contiguous amino acids of a FRMD4A polypeptide as defined herein, in particular, of a the human FRMD4A amino acid sequence as set forth in SEQ ID NO: 1.

In certain cases in accordance with this and other aspects of the present invention, the antibody molecule may bind to FRMD4A or a fragment thereof in a region that corresponds to the FERM domain of FRMD4A. For example, the antibody molecule may bind to FRMD4A in a region corresponding to or defined by the contiguous sequence of residues 20-322 of the amino acid sequence set forth in SEQ ID NO: 1. In certain preferred cases in accordance with this and other aspects of the present invention, the antibody molecule may bind to FRMD4A or a fragment thereof in the region defined by residues 63-83 or 1019-1039 of the sequence of SEQ ID NO: 1.

In certain cases in accordance with this and other aspects of the present invention the antibody molecule may be an anti-FRMD4A antibody molecule that tests positive as an inhibitor of SCC growth. This functional property of the antibody molecule may be assessed using any suitable assay, whether in vitro or in vivo. The antibody molecule may cause at least 10%, at least 20%, at least 30%, at least 40% or at least 50% reduction in the growth rate of cultured SCC cells in an in vitro cell growth assay as compared with cultured SCC cells grown under identical conditions, but in the absence of said antibody molecule. The cultured SCC cells may comprise primary SCC culture and/or an established SCC cell line (e.g. SCC13, SCC25 or SJG-15). The reduction in growth may be assessed over any suitable period, e.g. 24, 48, 96, 120 or more hours.

Binding kinetics and affinity (expressed as the equilibrium dissociation constant Kd) of the anti-FRMD4A antibody molecules may be determined using standard techniques, such as surface plasmon resonance, e.g. using BIAcore analysis.

An anti-FRMD4A antibody molecules may have a dissociation constant for ERMD4A of less than 50 nM, less than 40 nM, less than 30 nM, less than 20 nM, less than 10 nM, or less than 1 nM. For example, an antibody molecule may have an affinity for FRMD4A of 1 to 20 nM. Preferably antibody molecules of the present invention have affinity constants (K_{ID}) of less than 10 nM, more preferably less than 5 nM and most preferably less than 2 nM. The affinity constants for binding to FRMD4A or a peptide fragment of FRMD4A, e.g. the peptide consisting of the contiguous residues 63-83 or 1019-1039 of the amino acid sequence set forth as SEQ ID NO: 1, can be determined using techniques well known in the art such as Biacore SPR analysis.

Anti-FRMD4A antibody molecules may include any polypeptide or protein comprising an antibody antigen-binding site, including Fab, Fab2, Fab3, scFvs, diabodies, triabodies, tetrabodies, minibodies, intrabodies and single-domain antibodies, as well as whole antibodies of any isotype or sub-class. Antibody molecules and methods for their construction and use are described, in for example Holliger & Hudson, Nature Biotechnology 23(9): 1126-1136 (2005).

It has been shown that fragments of a whole antibody can perform the function of binding antigens. Thus reference to antibody molecule herein, and with reference to the methods, arrays and kits of the invention, covers a full antibody and also covers any polypeptide or protein comprising an antibody binding fragment. Examples of binding fragments are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment which consists of a VH domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site; (viii) bispecific single chain Fv dimers (WO 93/11161) and (ix) “diabodies”, multivalent or multispecific fragments constructed by gene fusion (WO94/13804; 58). Fv, scFv or diabody molecules may be stabilised by the incorporation of disulphide bridges linking the VH and VL domains. Minibodies comprising a scFv joined to a CH3 domain may also be made.

In some preferred embodiments, the anti-FRMNA antibody molecule may be a whole antibody. For example an IgG, IgA, IgE or IgM or any of the isotype sub-classes, particularly IgG1 and IgG4. The anti-FRMD4A antibody molecules may be monoclonal antibodies or polyclonal antibodies. Anti-FRMD4A antibody molecules may be chimeric, humanised or human antibodies.

Anti-FRMD4A antibody molecules as described herein may be isolated, in the sense of being free from contaminants, such as antibodies able to bind other polypeptides and/or serum components. Monoclonal antibodies are preferred for most purposes, though polyclonal antibodies may also be employed.

Methods of producing anti-FRMD4A antibody molecules include immunising a mammal (e.g. mouse, rat, rabbit, horse, goat, sheep or monkey) with the FRMD4A protein or a fragment thereof, e.g. a peptide fragment consisting of the contiguous sequence of amino acids 63-83 or 1019-1039 of the amino acid sequence set forth in SEQ ID NO: 1. Antibodies may be obtained from immunised animals using any of a variety of techniques known in the art, and screened, preferably using binding of antibody to antigen of interest. For instance, Western blotting techniques or immunoprecipitation may be used (Armitage et al., 1992, Nature 357: 80-82). Isolation of antibodies and/or antibody-producing cells from an animal may be accompanied by a step of sacrificing the animal.

As an alternative or supplement to immunising a mammal with a peptide, an antibody specific for a protein may be obtained from a recombinantly produced library of expressed immunoglobulin variable domains, e.g. using lambda bacteriophage or filamentous bacteriophage which display functional immunoglobulin binding domains on their surfaces. The library may be naive, that is constructed from sequences obtained from an organism which has not been immunised with any of the proteins (or fragments), or may be one constructed using sequences obtained from an organism which has been exposed to the antigen of interest.

In the present invention, the methods described in the examples may be employed to screen for further examples of anti-FRMD4A antibodies having antagonistic properties. After production and/or isolation, the biological activity of an anti-FRMD4A antibody molecule may be tested. For example, the ability of the antibody molecule to inhibit growth and/or metastasis of an SCC cell line or primary or secondary SCC tumour may be assessed in vitro or in vivo.

Antibody molecules normally comprise an antigen binding domain comprising an immunoglobulin heavy chain variable domain (VH) and an immunoglobulin light chain variable domain (VL), although antigen binding domains comprising only a heavy chain variable domain (VH) are also possible (e.g. camelid or shark antibodies). Such antibodies are included within the scope of the present invention.

Competition between antibody molecules may be assayed easily in vitro, for example using ELISA and/or by tagging a specific reporter molecule to one antibody molecule which can be detected in the presence of one or more other untagged antibody molecules, to enable identification of antibody molecules which bind the same epitope or an overlapping epitope. Such methods are readily known to one of ordinary skill in the art.

It is specifically contemplated herein that the anti-FRMD4A antibody of the present invention may be in the form of an intracellular antibody (“intrabody”). Intrabodies are antibodies that are directed against target molecules that are inside a cell and expressed within a particular cellular compartment as directed by the intracellular localization signals genetically fused to N- or C-terminus of the antibody. Intrabodies have wide applications in dissecting target protein function, in target validation and functional genomics, as well as acting as potential therapeutic reagents.

For a recent comprehensive review on intrabody technology, see Zhu and Marasco, Chapter 21 “Intracellular targeting of antibodies in mammalian cells”, pp. 573-587, in Gene Transfer and Expression in Mammalian Cells, S. C. Makrides (Ed.) 2003 Elsevier Science B. V., the entire contents of which are expressly incorporated herein by reference.

Without wishing to be bound by any particular theory, the present inventors believe that in addition to an accessible cell surface pool of FRMD4A there is in addition an intracellular pool of FRMD4A. As such, intrabodies may be preferred in some circumstances. Intrabodies may be produced by effecting intracellular expression of an antibody molecule, e.g. an scFv, in a mammalian cell. Expression may be effected by a gene therapy approach whereby a cell, e.g. a cancer cell such as an SCC cell, is targeted with a construct comprising an anti-FRMD4A antibody-encoding nucleic acid, thereby causing production of an anti-FRMD4A intrabody in the targeted cell. Strategies for production of intrabodies are known in the art, see for example, Shaki-Loewenstein et al., 2005, J. Immunol. Methods: 303(1-2): 19-39, the contents of which is expressly incorporated herein by reference.

### Antibody Conjugates

Alternatively or additionally, the antibody molecules of the present invention may be conjugated or linked to a therapeutically active moiety, for example a moiety that is cytotoxic. Such antibodies may be useful for targeting cancer that is spreading or prone to spread and delivering the therapeutically active moiety to cancer cells.

A further class of groups that can be incorporated into the antibodies of the present invention are affinity tags that can be introduced into the antibodies to enable them to be manipulated or detected in one or more subsequent steps. A wide range of affinity tags are known in the art suitable affinity tags include members of specific binding pairs, antibodies and antigens, biotin which binds to streptavidin and avidin, polyhistidine (e.g. hexa-His or tri-His tags) or amino di- or tri-carboxylates which bind to metal ions such as Ni²⁺ or Co²⁺, Flag or Glu epitopes which bind to anti-Flag antibodies, S-tags which bind to streptavidin, calmodulin binding peptide which binds to calmodulin in the presence of Ca2+; ribonuclease S which binds to aporibonuclease S; and c-Myc which recognises anti-c-Myc antibody. Examples of other affinity tags that can be used in accordance with the present invention will be apparent to those skilled in the art. Antibodies including these affinity tags can be easily purified and manipulated.

The term “therapeutically active moiety” encompasses a moiety having beneficial, prophylactic and/or therapeutic properties.

In one embodiment the therapeutically active moiety is a cytotoxic chemotherapeutic agent. Cytotoxic chemotherapeutic agents are well known in the art and include anti-cancer agents such as:

Alkylating agents including nitrogen mustards such as mechlorethamine (HN2), cyclophosphamide, ifosfamide, melphalan (L-sarcolysin) and chlorambucil; 10 ethylenimines and methylmelamines such as hexamethylmelamine, thiotepa; alkyl sulphonates such as busulfan; nitrosoureas such as carmustine (BCNU), lomustine (CCNLJ), semustine (methyl-CCN-U) and streptozoein (streptozotocin); and triazenes such as decarbazine (DTIC; dimethyltriazenoimidazolecarboxamide); Antimetabolites including folic acid analogues such as methotrexate (amethopterin); pyrimidine analogues such as fluorouracil (5-fluorouracil; 5-FU), floxuridine (fluorodeoxyuridine; FUdR) and cytarabine (cytosine arabinoside); and purine analogues and related inhibitors such as mercaptopurine (6-mercaptopurine; 6-MP), thioguanine (6-thioguanine; TG) and pentostatin (2′-deoxycoformycin). Natural Products including vinca alkaloids such as vinblastine (VLB) and vincristine; epipodophyllotoxins such as etoposide and teniposide; antibiotics such as dactinomycin (actinomycin D), daunorabicin (daunomycin; rubidomycin), doxorubicin, bleomycin, plicamycin (mithramycin) and mitomycin (mitomycin Q; enzymes such as L-asparaginase; and biological response modifiers such as interferon alphenomes. Miscellaneous agents including platinum coordination complexes such as cisplatin (cis-DDP) and carboplatin; anthracenedione such as mitoxantrone and anthracycline; substituted urea such as hydroxyurea; methyl hydrazine derivative such as procarbazine (N-methylhydrazine, MIH); and adrenocortical suppressant such as mitotane (o, p′-DDD) and aminoglutethimide; taxol and analogues/derivatives; and hormone agonists/antagonists such as flutamide and tamoxifen.

Methods of conjugating antibodies to therapeutic agents are well known in the art.

In a further embodiment, the cytotoxic moiety is a cytotoxic peptide or polypeptide moiety by which we include any moiety which leads to cell death.

Cytotoxic peptide and polypeptide moieties are well known in the art and include, for example, ricin, abrin, Pseudomonas exotoxin, RNase, tissue factor and the like.

The use of ricin as a cytotoxic agent is described in Burrows & Thorpe, P.N.A.S. USA 90: 8996-9000, 1993, incorporated herein by reference, and the use of tissue factor, which leads to localised blood clotting and infarction of a tumour, has been described by Ran et al., Cancer Res. 58: 4646-4653, 1998 and Huang et al., Science 275: 25 547-550, 1997. Tsai et al., Dis. Colon Rectum 38: 1067-1074, 1995 describes the abrin A chain conjugated to a monoclonal antibody and is incorporated herein by reference. Other ribosome inactivating proteins are described as cytotoxic agents in WO 96/06641. Pseudomonas exotoxin may also be used as the cytotoxic polypeptide moiety (see, for example, Aiello et al, P.N.A.S. USA 92: 10457-10461, 1995.

Certain cytokines, such as TNFα and IL-2, may also be useful as cytotoxic and/or therapeutic agents.

Certain radioactive atoms may also be cytotoxic if delivered in sufficient doses. Thus, the cytotoxic moiety may comprise a radioactive atom which, in use, delivers a sufficient quantity of radioactivity to the target site so as to be cytotoxic. Suitable radioactive atoms include phosphorus-32, iodine-125, iodine-131, indium-111, rhenium-186, rhenium-188 or yttrium-90, or any other isotope which emits enough energy to destroy neighbouring cells, organelles or nucleic acid. Preferably, the isotopes and density of radioactive atoms in the antibody of the invention are such that a dose of more than 4000 cGy, and more preferably at least 6000, 8000 or 10000 cGy, is delivered to the target site and, preferably, to the cells at the target site and their organelles, particularly the nucleus.

The radioactive atom may be attached to the binding moiety in known ways. For example, EDTA or another chelating agent may be attached to the binding moiety and used to attach 111In or 90Y. Tyrosine residues may be labelled with 125 I or 131I.

Alternatively, any of these systems can be incorporated into a prodrug system. Such prodrug systems are well known in the art and include ADEPT systems in which an antibody according to the present invention is conjugated or conjugatable or fused to an agent capable of converting a prodrug to a cytotoxic moiety is an enzyme for use in antibody directed enzyme prodrug therapy.

### Medical Uses

The antagonists of FRMD4A and/or the Hippo pathway find use in the treatment of proliferative disorders, particularly cancer, in a mammalian subject. Preferably the antagonist of FRMD4A comprises an anti-FRMD4A antibody molecule as defined herein.

Preferably the cancer is an SCC or (other) epithelial cancer. The mammalian subject is preferably a human. The subject may have been diagnosed as having or being susceptible to developing a cancer, such as an SCC. In some cases the subject may have had surgical and/or pharmaceutical treatment for a cancer, including SCC or (other) epithelial cancer (e.g. the treatment in accordance with the present invention may be of a subject who or that has had surgical resection of an SCC tumour).

The cancer may comprise a carcinoma (e.g. an SCC) of a tissue or organ selected from: epithelial tissue, skin, oral cavity, tongue, head, neck, lips, mouth, oesophagus, urinary bladder, prostate, lung, vagina, cervix, kidney, thyroid, mammary papilla, breast, liver and colon. In certain cases, the cancer may be SCC of the head and neck (HNSCC).

Without wishing to be bound by any theory, the present inventors believe that the antagonists of the invention, such as the anti-FRMD4A antibody molecules defined herein, produce beneficial effects on a mammalian subject having a cancer, including an SCC by virtue of one or more effects selected from:
(i) a decrease in the rate of growth;
(ii) an increase in apoptosis;
(iii) an increase in the cellular differentiation;
(iv) a decrease in metastasis; and
(v) a decrease in the invasion,
   of one or more tumour cells, e.g. one or more SCC tumour cells.

Additionally or alternatively the method of treating may comprise reducing the number of cancer stem cells in a tumour, including an SCC tumour, e.g. by direct cell killing or by inducing developmental changes in the cancer stem cells towards non-stem cell phenotype (such as inducing differentiation of cancer stem cells). Cancer stem cells are frequently responsible for sub-optimal outcome of anti-cancer treatment strategies, e.g. relapse following treatment of a tumour. Therefore, the present inventors believe that methods of targeting cancer stem cells, including SCC stem cells, as provided by the present invention may offer significant advantage over conventional therapeutic strategies.

### Pharmaceutical Compositions

The anti-FRMD4A antibody molecules of the present invention may be comprised in pharmaceutical compositions with a pharmaceutically acceptable excipient.

A pharmaceutically acceptable excipient may be a compound or a combination of compounds entering into a pharmaceutical composition which does not provoke secondary reactions and which allows, for example, facilitation of the administration of the anti-FRMD4A antibody molecule, an increase in its lifespan and/or in its efficacy in the body or an increase in its solubility in solution. These pharmaceutically acceptable vehicles are well known and will be adapted by the person skilled in the art as a function of the mode of administration of the anti-FRMD4A antibody molecule.

In some embodiments, anti-FRMD4A antibody molecules may be provided in a lyophilised form for reconstitution prior to administration. For example, lyophilised antibody molecules may be re-constituted in sterile water and mixed with saline prior to administration to an individual.

Anti-FRMD4A antibody molecules will usually be administered in the form of a pharmaceutical composition, which may comprise at least one component in addition to the antibody molecule. Thus pharmaceutical compositions may comprise, in addition to the anti-FRMD4A antibody molecule, a pharmaceutically acceptable excipient, carrier, buffer, stabilizer or other materials well known to those skilled in the art. Such materials should be non-toxic and should not interfere with the efficacy of the anti-FRMD4A antibody molecule. The precise nature of the carrier or other material will depend on the route of administration, which may be by bolus, infusion, injection or any other suitable route, as discussed below.

For intra-venous administration, e.g. by injection, the pharmaceutical composition comprising the anti-FRMD4A antibody molecule may be in the form of a parenterally acceptable aqueous solution which is pyrogen-free and has suitable pH, isotonicity and stability. Those of relevant skill in the art are well able to prepare suitable solutions using, for example, isotonic vehicles, such as Sodium Chloride Injection, Ringer's Injection, Lactated Ringer's Injection. Preservatives, stabilizers, buffers, antioxidants and/or other additives may be employed as required including buffers such as phosphate, citrate and other organic acids; antioxidants, such as ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens, such as methyl or propyl paraben; catechol; resorcinol; cyclohexanol; 3′-pentanol; and m-cresol); low molecular weight polypeptides; proteins, such as serum albumin, gelatin or immunoglobulins; hydrophilic polymers, such as polyvinylpyrrolidone; amino acids, such as glycine, glutamine, asparagines, histidine, arginine, or lysine; monosaccharides, disaccharides and other carbohydrates including glucose, mannose or dextrins; chelating agents, such as EDTA; sugars, such as sucrose, mannitol, trehalose or sorbitol; salt-forming counter-ions, such as sodium; metal complexes (e.g. Zn-protein complexes); and/or non-ionic surfactants, such as TWEEN™, PLURONICS™ or polyethylene glycol (PEG).

A pharmaceutical composition comprising an anti-FRMD4A antibody molecule may be administered alone or in combination with other treatments, either simultaneously or sequentially dependent upon the condition to be treated.

An anti-FRMD4A antibody molecule as described herein may be used in a method of treatment of the human or animal body, including prophylactic treatment (e.g. treatment before the onset of a condition in an individual to reduce the risk of the condition occurring in the individual; delay its onset; or reduce its severity after onset). The method of treatment may comprise administering an anti-FRMD4A antibody molecule to an individual in need thereof.

Administration is normally in a “therapeutically effective amount”, this being sufficient to show benefit to a patient. Such benefit may be at least amelioration of at least one symptom. The actual amount administered, and rate and time-course of administration, will depend on the nature and severity of what is being treated, the particular mammal being treated, the clinical condition of the individual patient, the cause of the disorder, the site of delivery of the composition, the method of administration, the scheduling of administration and other factors known to medical practitioners. Prescription of treatment, e.g. decisions on dosage etc, is within the responsibility of general practitioners and other medical doctors and may depend on the severity of the symptoms and/or progression of a disease being treated. Appropriate doses of antibody molecules are well known in the art (Ledermann J. A. et al. (1991) Int. J. Cancer 47: 659-664; Bagshawe K. D. et al. (1991) Antibody, Immunoconjugates and Radiopharmaceuticals 4: 915-922). Specific dosages may be indicated herein or in the Physician's Desk Reference (2003) as appropriate for the type of medicament being administered may be used. A therapeutically effective amount or suitable dose of an antibody molecule may be determined by comparing its in vitro activity and in vivo activity in an animal model. Methods for extrapolation of effective dosages in mice and other test animals to humans are known. The precise dose will depend upon a number of factors, including whether the antibody is for prevention or for treatment, the size and location of the area to be treated, the precise nature of the antibody (e.g. whole antibody, fragment) and the nature of any detectable label or other molecule attached to the antibody.

A typical antibody dose will be in the range 100 μg to 1 g for systemic applications, and 1 μg to 1 mg for topical applications. An initial higher loading dose, followed by one or more lower doses, may be administered. Typically, the antibody will be a whole antibody, e.g. the IgG1 or IgG4 isotype. This is a dose for a single treatment of an adult patient, which may be proportionally adjusted for children and infants, and also adjusted for other antibody formats in proportion to molecular weight. Treatments may be repeated at daily, twice-weekly, weekly or monthly intervals, at the discretion of the physician. Treatments may be every two to four weeks for subcutaneous administration and every four to eight weeks for intra-venous administration. Treatment may be periodic, and the period between administrations is about two weeks or more, e.g. about three weeks or more, about four weeks or more, or about once a month. Treatment may be given before, and/or after surgery, and/or may be administered or applied directly at the anatomical site of surgical treatment or invasive procedure. Suitable formulations and routes of administration are described above.

In some preferred embodiments, the therapeutic effect of the anti-FRMD4A antibody molecule may persist for several half-lives, depending on the dose. For example, the therapeutic effect of a single dose of anti-FRMD4A antibody molecule may persist in an individual for 1 month or more, 2 months or more, 3 months or more, 4 months or more, 5 months or more, or 6 months or more.

### EXAMPLES

### Example 1

### Antagonists of FRMD4a In Vitro and In Vivo

### Materials and Methods

### Human Tissue Specimens

Appropriate informed consent was obtained from patients diagnosed with oral SCC, prior to operation at Addenbrooke's Hospital. Small biopsy specimens were removed from freshly resected oral SCCs by a Consultant Pathologist, before fixation. Normal specimens of skin were obtained with informed consent from operations to remove excess skin. Specimens were processed as described below.

### In-Situ Hybridisation

Probes were generated to FRMD4A and beta-actin as a control, then In situ hybridization was performed as previously described on sections of paraffin-fixed human foreskin.

### Antibodies

Six polyclonal antibodies to FRMD4A were produced and purified (SGO-1 to -6). Polyclonal antibodies were generated using the following peptides from the FRMD4A protein as antigen. SGO-1 and SGO-2 were raised against a peptide corresponding to amino acids 63-83 (SEQ ID NO: 3: IAFTDETGHLNWLQLDRRVLE). SGO-3 and SGO-4 were raised against a peptide corresponding to amino acids 568-588 (SEQ ID NO: 4: PHKGLPPRPPSHNRPPPPQSL). SGO-5 and SGO-6 were raised against a peptide corresponding to amino acids 1019-1039 (SEQ ID NO: 5: ATENSPILDGSESPPHQSTDE). Rabbits were immunised with the respective peptides and serum collected prior to purification of the FRMD4A polyclonal antibody using affinity purification on columns with bound antigen attached to pull out the FRMD4A specific antibodies.

The following antibodies were also used, and were obtained from commercial sources unless specified otherwise.

Alpha-6 integrin [GOH3] (BD Pharmigen, Oxford, UK)

### E-cadherin [HECD-1]

### Anti-GFP

### Ki67

Cleaved caspase-3

### Vimentin [VI-01] (Abcam, Cambridge, UK)

### Snail (Abcam, Cambridge, UK)

### YAP1 (Abcam, Cambridge, UK)

### Lats1 (Abcam, Cambridge, UK)

### MST1 (Abcam, Cambridge, UK)

### Involucrin [SY3]

### CD44 (BD Pharmigen, Oxford, UK)

### GAPDH

AlexaFluor-488 or -555 conjugated secondary antibodies were obtained from Invitrogen, Corp.

### Anti-rabbit HRP

### Anti-mouse HRP

### Histology, Immunohistochemistry, and Immunofluorescence

For sections, tissue was either fixed in 10% neutral-buffered formalin and embedded in paraffin or frozen in OCT embedding matrix (Raymond A Lamb, UK). Paraffin sections underwent epitope retrieval by boiling in citrate buffer for 10 minutes. Blocking buffer contained 10% fetal calf serum, 4% bovine serum, 2.5% fish skin gelatin and 0.05% Tween 20. Ki67 sections were photographed and analyzed using the Ariol SL-50 system (Applied Imaging, Corp.). Immunofluorescence slides were imaged using the Leica Tandem Confocal microscope.

### Western Blotting

Cells were trypsinised and pelleted by centrifugation. Protein lysates were prepared in RIPA buffer containing protease and phosphatase inhibitors. After the addition of Laemmli buffer, samples were boiled for 5 minutes, and resolved on 4% to 12% gradient polyacrylamide gels. Separated proteins were transferred to nitrocellulose membrane and blocked with a TTM buffer consisting of 2.5% skimmed milk powder and 0.05% Tween 20. Primary antibodies to FRMD4A and YAP were used at a 1:200 concentration; GAPDH was used for loading control at 1:1000 concentration.

### Cell Culture and Lentivirus Infection

SCCs were cultured without a fibroblast feeder layer in calcium-containing FAD medium (three parts DMEM medium and one part F12 medium supplemented with 1.8×10⁻⁴ mol/L adenine) supplemented with 10% FCS, hydrocortisone, insulin, cholera toxin, and epidermal growth factor.

The YFP/Luciferase lentivirus construct was a kind gift from Dr Scott Lyons. Virus was produced by transiently infecting 293T cells using the second generation packaging system. Infected cells were selected using hygromycin.

shRNA constructs were obtained from Open Biosystems, and virus was produced by transiently infecting 293T cells using the third generation packaging system. Infected cells were selected using puromycin.

### Colony Forming Assays and Incucyte Assays

For colony forming assays, 100 cells were plated in triplicate in 10 cm plates. After 14 days, cultures were fixed and stained with 1% rhodamine B and 1% Nile blue (Acros Organic). Colony-forming efficiency was defined as the percentage of plated cells that formed a colony of three or more cells.

For Incucyte assays, 1000 cells were plated in triplicate for each condition, in a 48-well plate. Plates were imaged every 3 hours for 7 days.

### Xenografts

All mice used were NOD.Cg-Prkdc^{scid} I12rg^{tm1Wj1}/SzJ or NOD/SCID Gamma (NSG) as they are commonly known. Mice were originally purchased from the Jackson Laboratories (Maine, USA) and then bred in-house. Experiments were subject to Cancer Research UK ethical review and were performed under the terms of a U.K. Government Home Office license. Tongue xenografts were carried out using inhalation anaesthesia and injection into the mucosa with a 30 g needle and 1 ml syringe. Mice were grafted using inhalation anaesthesia. An area of skin on the back of each mouse was shaved and cleaned with a betadine solution. Using a 6 mm punch biopsy punch a full thickness wound was created, into which the silicone chamber was inserted and stapled in place. A preparation of cells could then be injected into the chamber. After one week the top of the silicone chamber was cut off to allow the tumour to develop exposed to the normal skin-air interface. After a further week the whole silicone chamber was carefully removed. Mice were scanned with the Xenogen IVIS following an i.p. injection of luciferin (10 μl/g).

### Antibody Treatment

Mice were tongue grafted with 1×10E5 SCC25 cells. After one week an initial measurement was made with the Xenogen IVIS. Following this, each mouse was i.p injected with either SGO-1, SGO-4 or an IgG control (WIB10). Measurements were made weekly with the Xenogen IVIS.

### Hyaluronic Acid Treatment

Mice were grafted into the back skin with 1×10E6 SCC13 cells.

After one week an initial measurement was made with the Xenogen IVIS. Following this, each mouse was directly injected into the tumour with 200 μl of 0.1% HA. Measurements were made weekly with the Xenogen IVIS, followed by an intra-tumour injection of HA.

### 17-DMAG Treatment

Mice were tongue grafted with 1×10E5 SCC25 cells. After one week an initial measurement was made with the Xenogen IVIS. Following this, each mouse was i.p injected with either 17-DMAG or vehicle control. Measurements were made weekly with the Xenogen IVIS, followed by a repeat injection with either 17-DMAG or vehicle control. The first five weekly doses were 0.02 mg per mouse, this was increased to 0.04 mg for the remainder of the experiment.

### Results

### FRMD4A is a Marker of Basal Cells in Interfollicular Epidermis (IFE) and is Lost During Differentiation

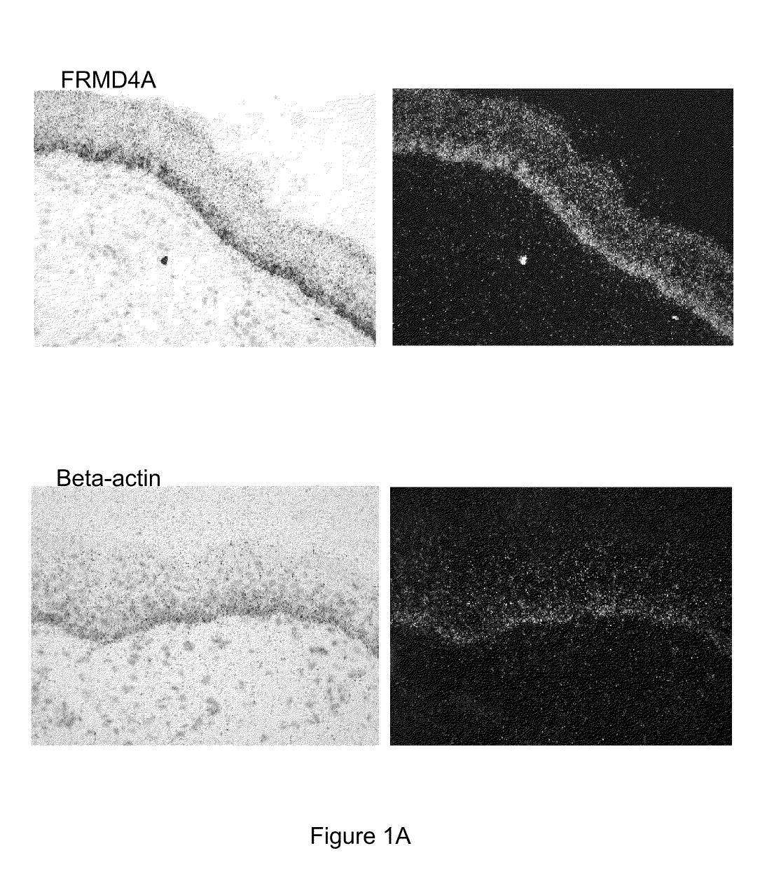

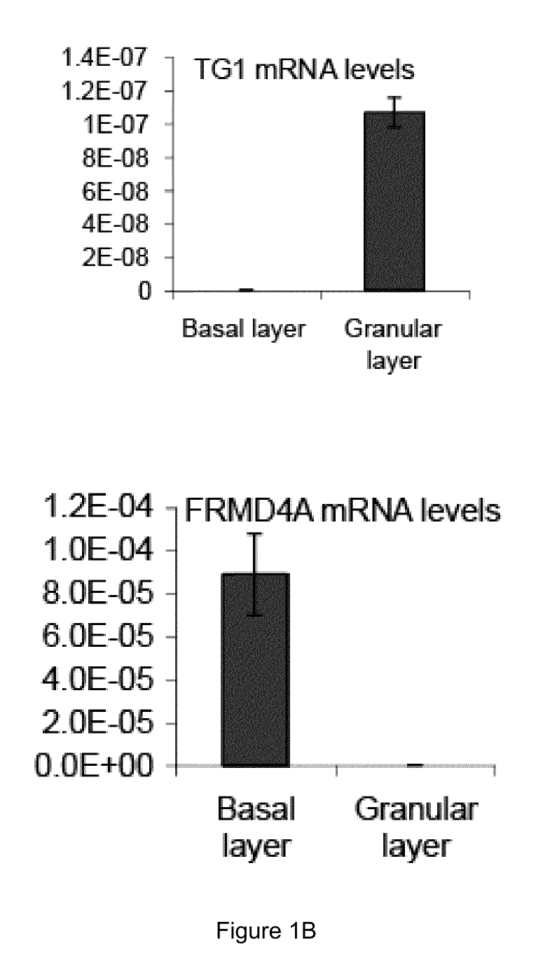

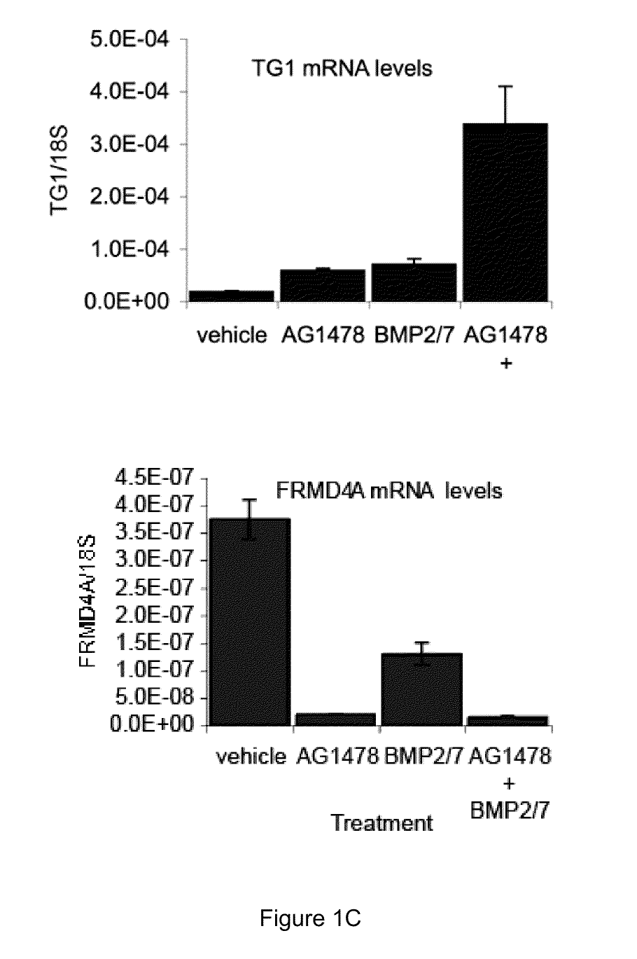

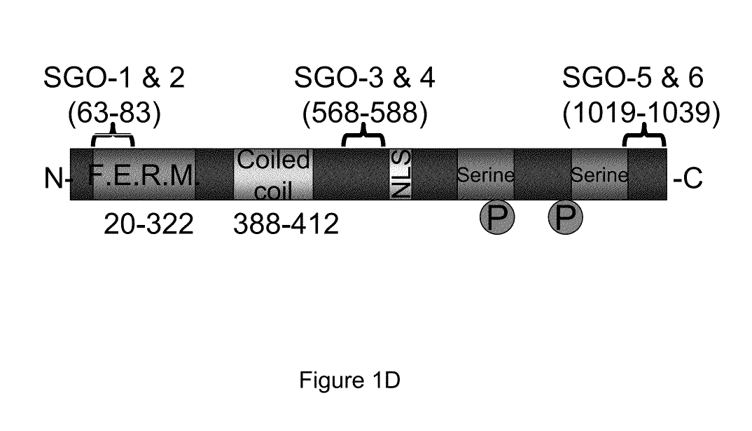

To determine the expression of FRMD4A mRNA in normal human skin we carried out in-situ hybridisations on normal human foreskin. Strong signal for FRMD4A was seen in the basal layer of the epidermis, which was lost immediately suprabasally (FIG. 1A). Using the Zeiss PALM Laser Capture Microdissection system, samples were collected separately from the basal and granular layers of human abdomen skin sections. Levels of mRNA expressed relative to 18S showed almost absent levels of the differentiation marker transglutaminase (TG-1) in the basal sample, with high levels as expected in the granular layer. Levels of FRMD4A were contrary to this, with high levels in the less differentiated basal layer, which dropped to almost nothing in the more differentiated granular layer (FIG. 1B). Similarly, normal human keratinocytes grown in serum free culture were induced to differentiate, by the addition of AG1478 and BMP2/7 (FIG. 1C). Differentiation of the keratinocytes was confirmed by increased levels of transglutaminase in the presence of these agents alone compared to treatment with vehicle alone. Addition of both together led to expression of even higher levels of the differentiation marker TG-1. FRMD4A levels were high in the vehicle treated sample, but became barely detectable in the differentiated samples (FIG. 1C) showing that FRMD4A is associated with less differentiated cell types.

Commercial antibodies to FRMD4A are not currently available so were developed in-house. In total six polyclonal antibodies to FRMD4A were purified and tested; two separate polyclonals were generate to each of the three separate peptides shown in FIG. 1D.

SGO-1 and SGO-2 were generated to a peptide consisting of the contiguous residues 63-83 of the human FRMD4A protein sequence set forth in SEQ ID NO: 1. This region of the FRMD4A sequence lies within a FERM domain (residues 20-322).

SGO-3 and SGO-4 were generated to a peptide consisting of the contiguous residues 568-588 of the human FRMD4A protein sequence set forth in SEQ ID NO: 1.

SGO-5 and SGO-6 were generated to a peptide consisting of the contiguous residues 1019-1039 of the human FRMD4A protein sequence set forth in SEQ ID NO: 1.

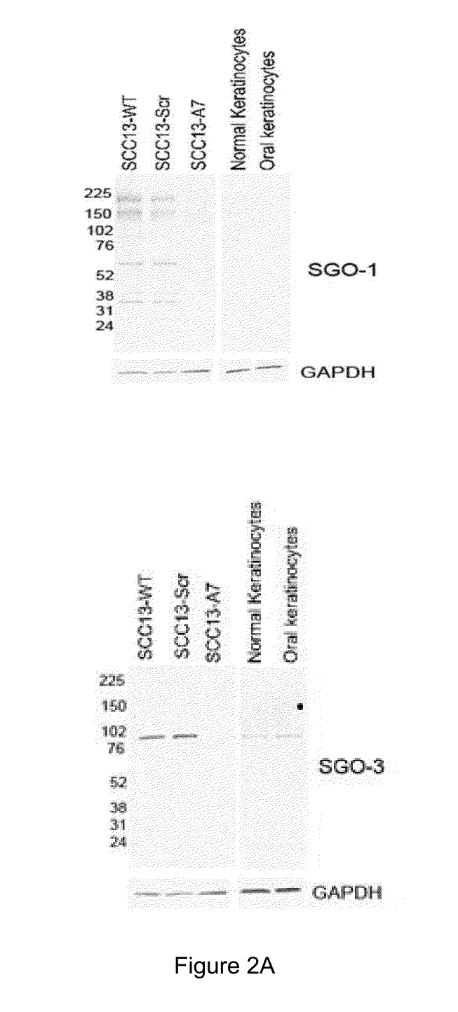

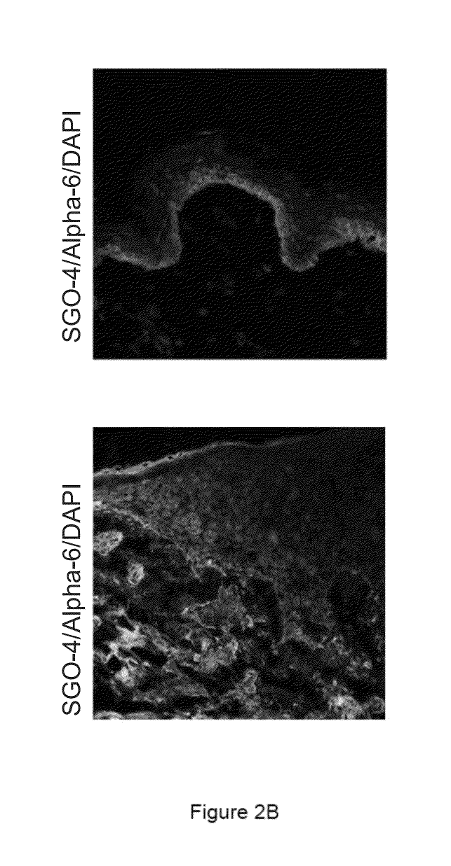

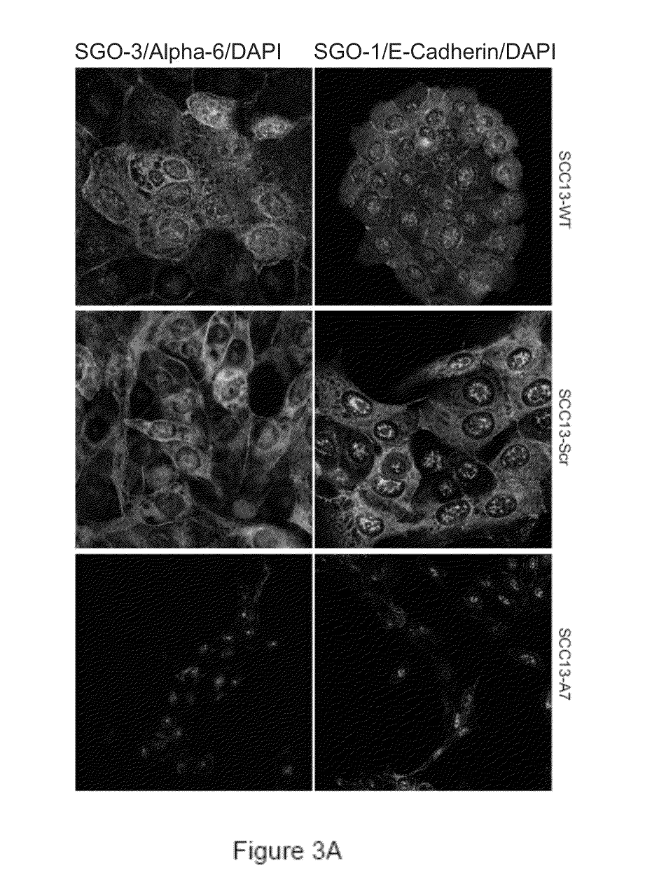

In order to test the specificity of the antibodies to FRMD4A, SCC lines were infected with commercial lentivirus shRNAs to FRMD4A. Cells were also infected with a scrambled sequence as a control. The antibodies to FRMD4A were shown to be specific as the protein detected by the antibodies was lost in the cells where FRMD4A has been knocked down (FIG. 2A). Knockdown of FRMD4A using five different FRMD4A specific oligo sequences was confirmed at the protein level by western blot (data not shown). The blots also compare levels of FRMD4A in SCCs compared to keratinocytes from normal human skin and oral mucosa. FRMD4A in the normal skin and oral mucosa keratinocytes is much lower than in the SCCs. Expression of FRMD4A predominantly in the basal layer of normal skin was confirmed by immunofluorescence staining of normal human abdomen skin. Staining is present continuously along the basal layer. Tumour sections from tumour biopsy samples were also stained (FIG. 2B). FRMD4A was seen in all twelve tumours, but to varying degrees.

### FRMD4a Influences Cell Shape and Cell-Cell Interaction

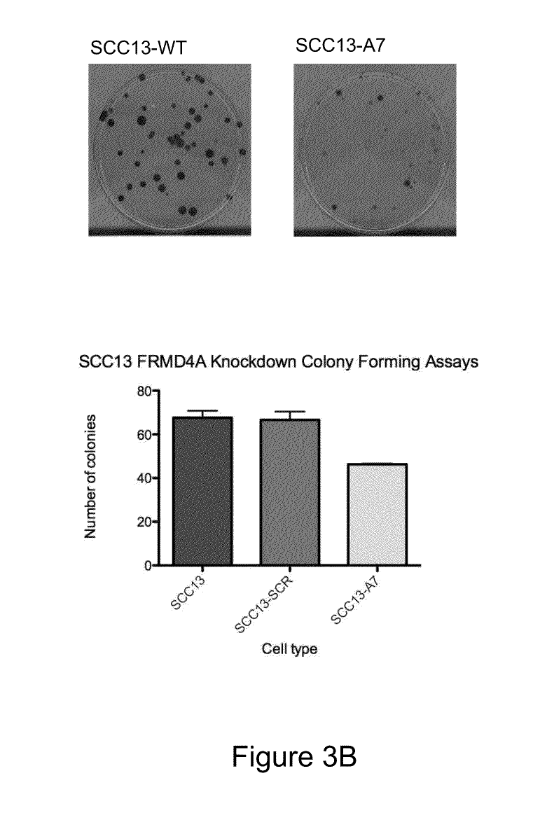

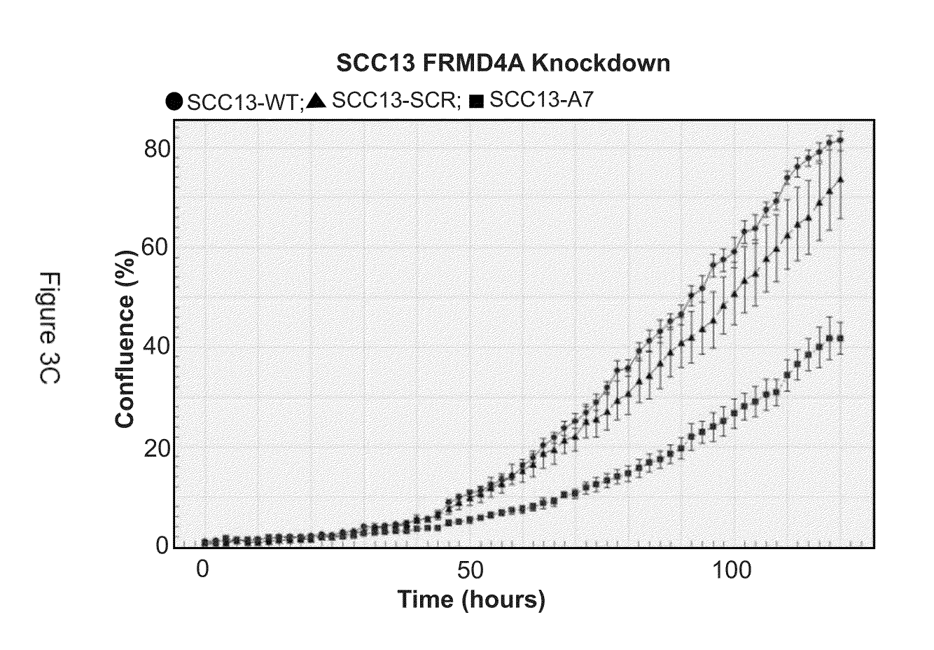

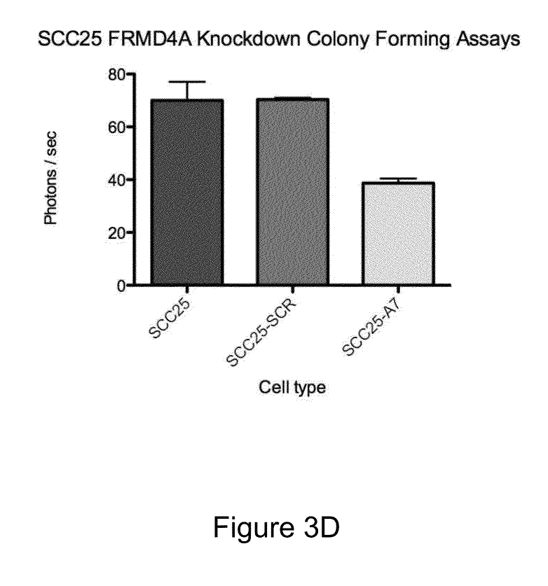

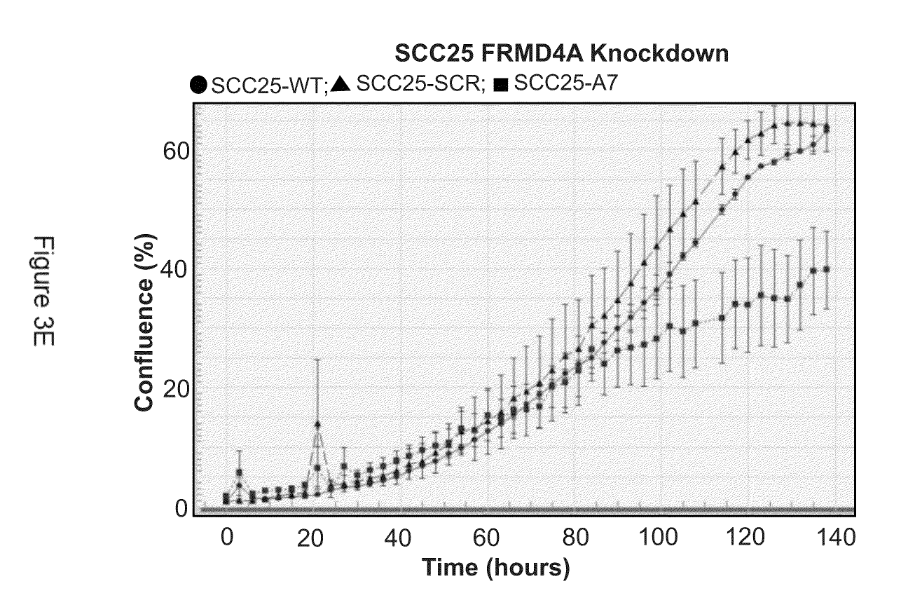

SCC cell lines grown in culture were infected with commercial lentivirus shRNAs to FRMD4A. Cells were then plated on coverslips and immunostained. SCC13 Wild-type (SCC13-WT) and SCC13-scrambled (SCC13-SCR) show FRMD4A staining mainly in the cytoplasm, but with some specific staining also localised to the cell borders and the nucleus. Cells were counterstained with the cell surface adhesion molecule e-cadherin to demonstrate cell-cell interaction (FIG. 3A). SCC13-FRMD4A knockdown cells (SCC13-A7) have an absence of FRMD4A and show a loss of e-cadherin at their cell borders (FIG. 3A). The general morphology of the knockdown cells has changed to a more spindle like phenotype with less cell-cell contact. In order to investigate this further, cells were imaged using the time-lapse Incucyte microscope system. Movies generated show SCC13-WT and SCC13-SCR cells form regular colonies, while SCC13-A7 cells avoid contact with each other until they are forced together by bulk of numbers.

### Knockdown of FRMD4a Reduces Growth In Vitro and Cancer Stem Cell Number

Colony forming efficiency of SCCs in culture can be used as a surrogate to determine the percentage of cancer stem cells in a given population. A reduction in colony forming efficiency was seen in both SCC13 (FIG. 3B) and SCC25 cells (FIG. 3D) when FRMD4A was knocked down. Overall growth of the knocked down SCC cell lines was reduced by approximately 50% as shown by Incucyte cell proliferation experiments (FIGS. 3C and 3E).

### Orthotopic Xenograft Models of Human SCCs

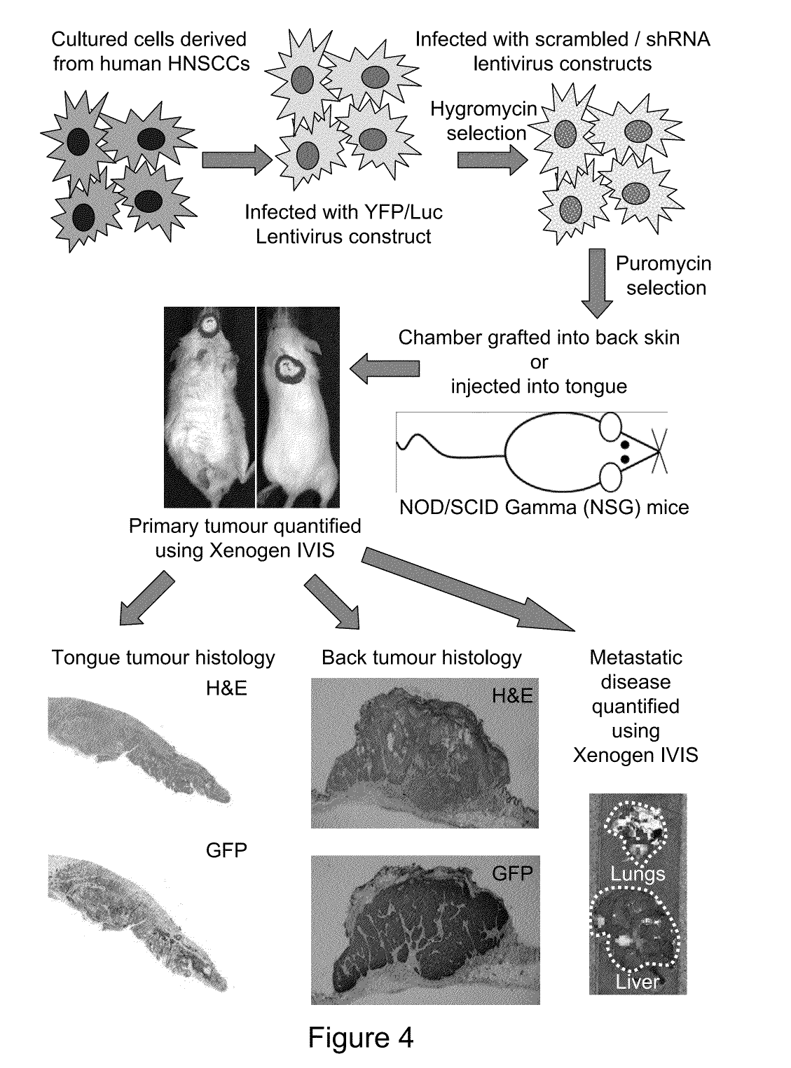

Previous attempts at emulating human SCCs using xenografts have not adequately replicated the human disease and so we attempted to improve on this for in vivo experiments. Cultured cells were first infected with a lentivirus construct that expressed a YFP/luciferase fusion protein, before antibiotic selection and secondary infection with shRNAs to FRMD4A. These were then either injected into a silicone chamber surgically implanted under the back skin of the mouse or directly injected into the tongue. All xenografts were made using NOD/SCID Gamma (NSG) mice, as they are severely immunocompromised and have been shown to take on grafts readily. As a result of the infection with luciferase, development of the primary tumours was measured using the Xenogen In-vivo imaging system (IVIS), following i.p. injection with luciferin. Organs were scanned post-mortem to detect metastatic disease. A summary of the protocols used in the xenograft experiments is shown in FIG. 4.

Interesting variations on the tumour developing ability of SCCs were seen depending on the location of the graft: chamber grafted SCCs required a minimum of 1×10E5 cells in order to successfully form a tumour, whereas tongue grafted tumours developed after injection of only 100 cells. Knockdown of FRMD4A increased the number of cells required to form a xenograft tumour with SCC25, from 100 cells to 1×10E5 cells (data not shown). The requirement for additional cells to be able to form a xenograft tumour is often indicative of a detrimental effect on, or loss of cancer stem cells from the injected cell population.

### Stable Knockdown of FRMD4A Reduces the Growth Rate of Human SCCs and Increases Survival

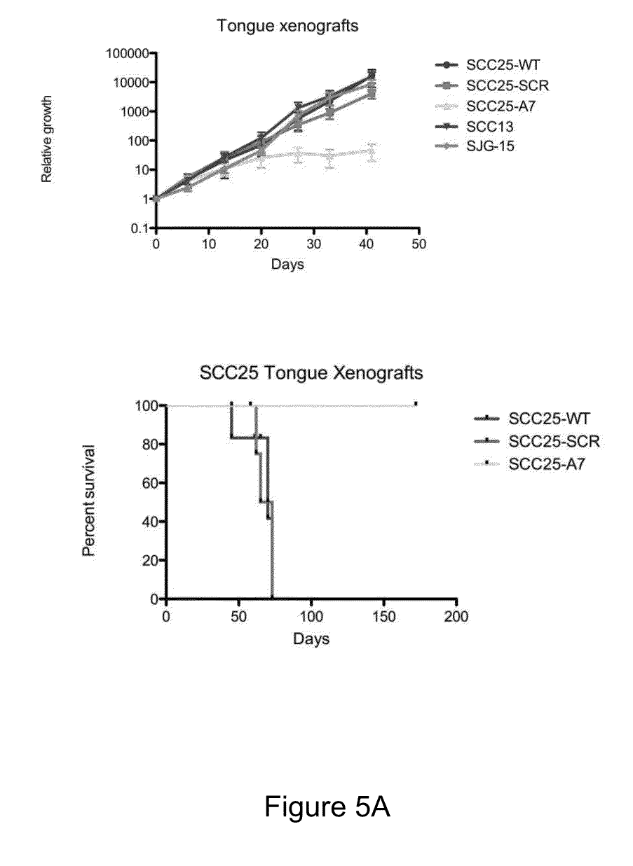

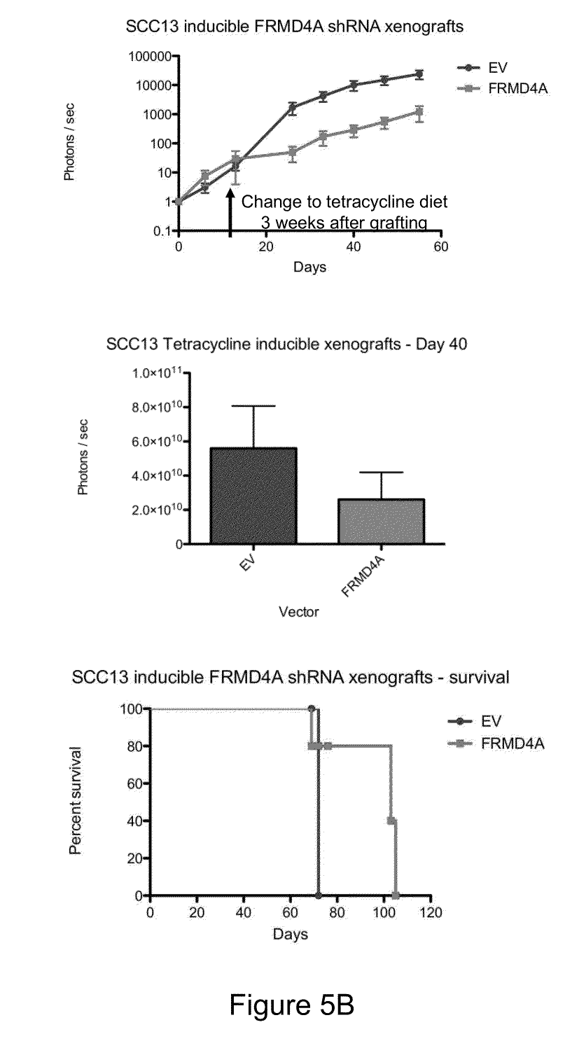

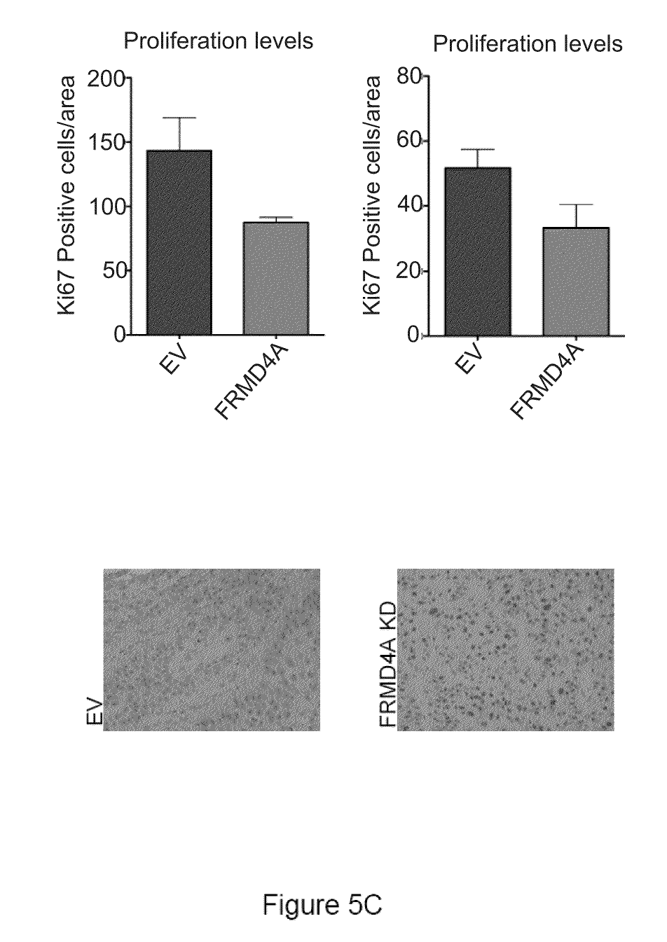

Tongue xenografts of cell lines SCC25-WT, SCC25-SCR and SCC25-A7 were measured using the Xenogen IVIS. These lines were compared with xenografts with SCC13 and SJG-15 (an aggressive cell line recently derived from a tongue SCC). Growth of the FRMD4A knockdown tumours was seen to be considerably slower than that of other lines, in keeping with in vitro experiments (FIG. 5A). Mice were culled when their weight dropped by 20% of their pre-grafting weight or if their condition deteriorated generally. Survival data for SCC25-WT, SCC25-SCR and SCC25-A7 grafted mice showed that knocking down FRMD4A increased the lag time before culling was required by more than 200% (FIG. 5A).

### Inducible Knockdown of FRMD4A Reduces the Growth Rate of Human SCCs and Increases Survival

SCC13 cell lines were infected with a “Tet-on” doxyxcycline inducible shRNAs or control empty vector (EV), and then injected into chamber grafts on the back of NSGs. The chambers were removed after two-weeks and the diet changed to doxyxcycline-rich chow after a further week. Prior to the change of diet, both arms of the experiment grew steadily, with the FRMD4A-shRNA tumours growing marginally faster. Following the addition of doxyxcycline to the diet, the EV tumours grew at a steady rate, while the FRMD4A-shRNA tumours grew significantly slower (FIG. 5B). In order to adhere to animal licensing laws, mice were culled when tumours grew to a maximum diameter of 15 mm in any orientation. At the time of culling the first animal the EV group had a higher total tumour size compared to the FRMD4A-shRNA group (FIG. 5B). Survival was demonstrably longer in the FRMD4A knockdown group (FIG. 5B).

### FRMD4A Knockdown Reduces Proliferation and Induces Apoptosis

To better explain the differences in growth rates of the primary tumours, histological sections were stained for the proliferation marker Ki67. Sections were then scanned and quantified using the Ariol SL-50 system. FRMD4A knockdown tumours showed a decrease in the numbers of Ki67 positive cells in relation to both the area of the tumour section and the total number of proliferating cells (FIG. 5C). The FRMD4A knockdown tumours also stained positive for the apoptosis marker cleaved caspase-3 at greater levels than in comparison to control EV tumours (FIG. 5C).

### Knockdown of FRMD4A Reduces Invasion and Metastases

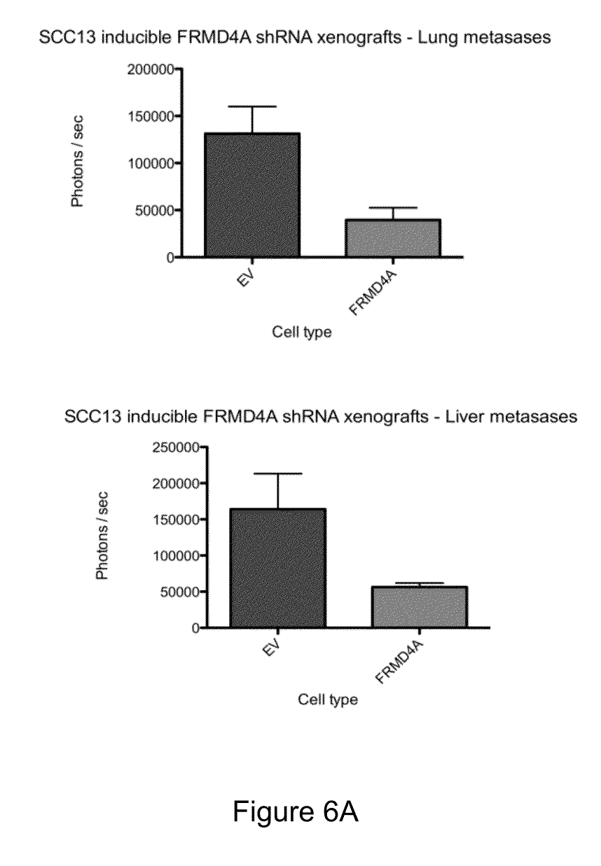

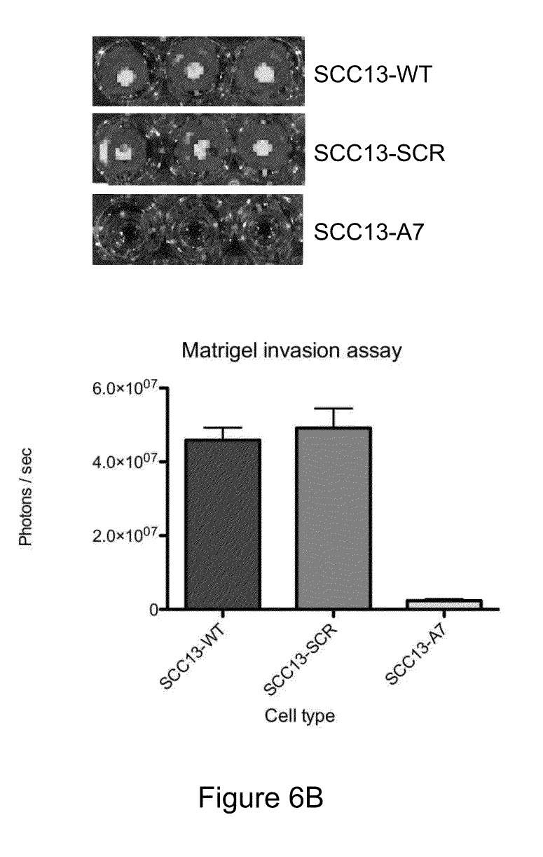

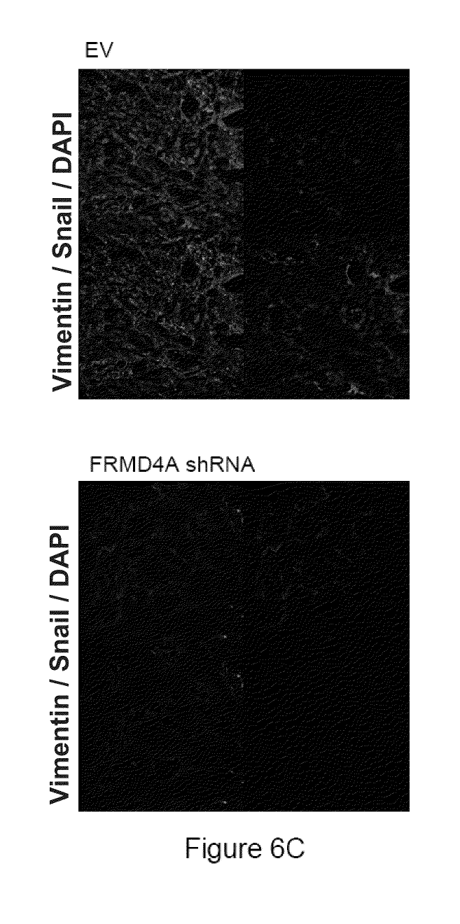

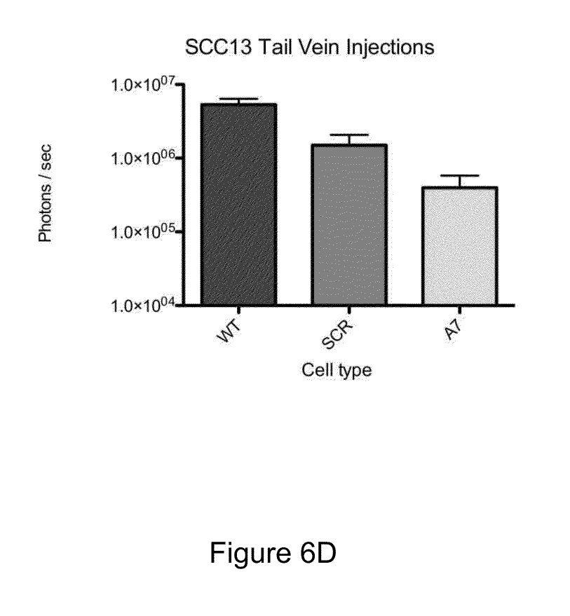

Organs from each mouse in the doxyxcycline-inducible experiment were scanned immediately post-mortem. Lungs and liver were selected for quantification, as they are the organs most affected by metastases in the human disease. In both the lungs and the livers, greater levels of metastastic disease where found in the empty vector (EV) control group (FIG. 6A) versus the FRMD4A knockdown group.

Sections of the primary tumours were stained with recognised markers predicting metastatic propensity of tumours. Levels of SNAIL, the zinc finger transcription factor and vimentin, the intermediate filament protein, were present in the empty vector control (EV) tumours, but greatly reduced in the FRMD4A-shRNA knock down tumours (FIG. 6c).

In order to test the affect on the invasive abilities of SCCs in vitro, the cells were serum starved for 24 hrs before being added to Boyden chambers. Serum-free medium in the chamber and serum-rich medium in the well below created a gradient across the membrane. After 24 hrs the matrigel was removed and the degree of invasion quantified by adding luciferin and scanning with the Xenogen IVIS. Cells did invade in all groups, however, a clearly visible and quantifiably less amount of SCC13-A7 cells invaded compared to the SCC13-WT and SCC13-SCR control groups (FIG. 6B).

To further investigate the mechanism by which FRMD4A knockdown inhibits metastasis, cells from each group were tail vein injected into the bloodstream of NSG mice. Seven days later the mice were culled and their lungs scanned in order to quantify the level of surviving SCC cells. It was assumed that by seven days cells would have migrated out of the capillaries and therefore this is a true reflection of metastasised cells, rather than merely cells sticking in the narrow capillaries of the lungs. SCC13-WT and SCC13-SCR controls showed higher levels of surviving cells in the lungs, compared to SCC13-A7 cells with FRMD4A knocked down (FIG. 6D).

### FRMD4A Knockdown Induces Differentiation

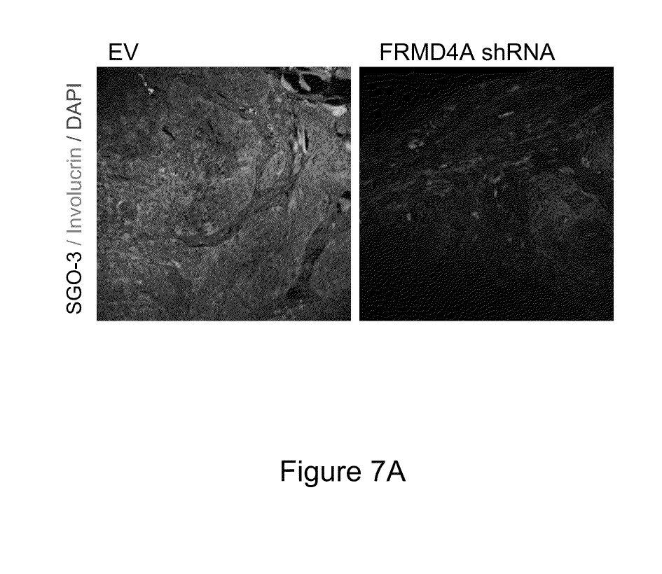

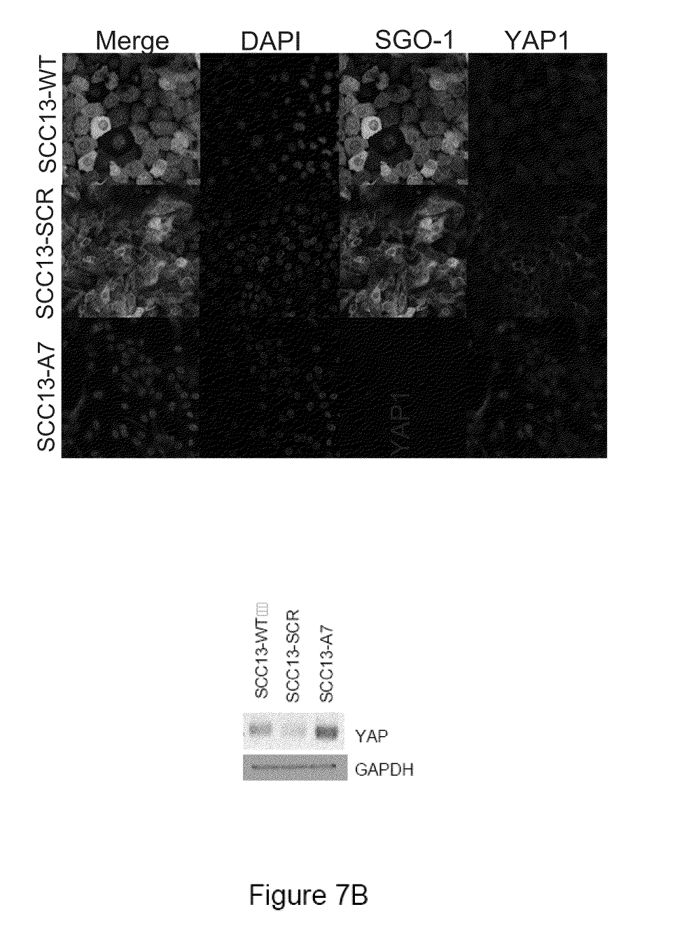

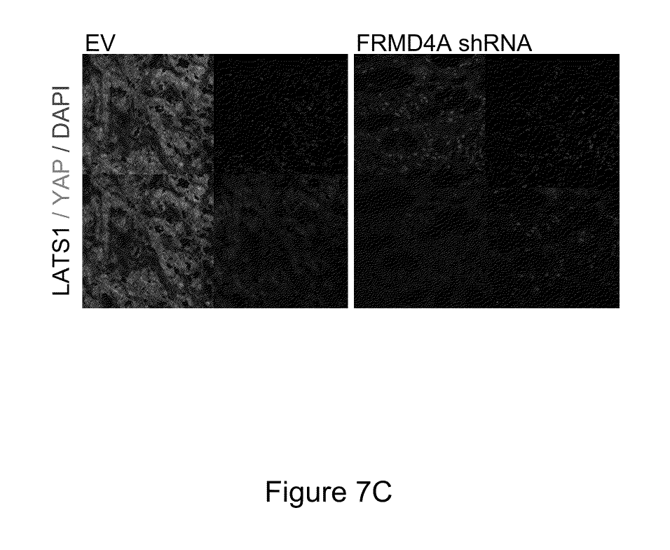

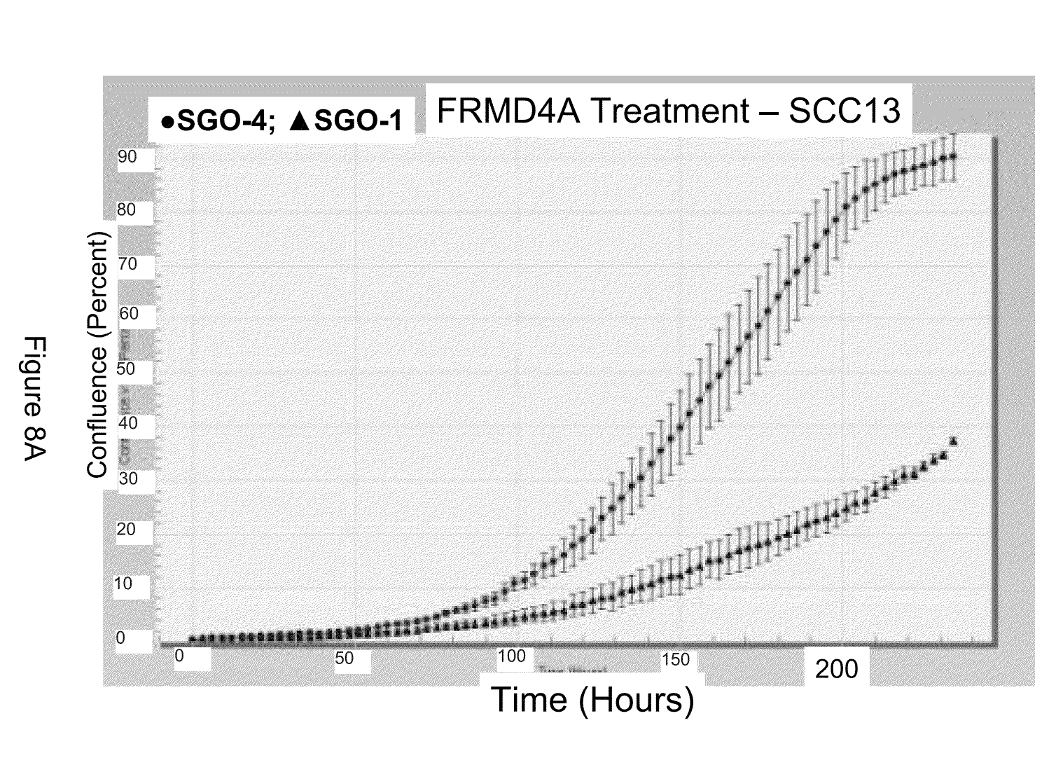

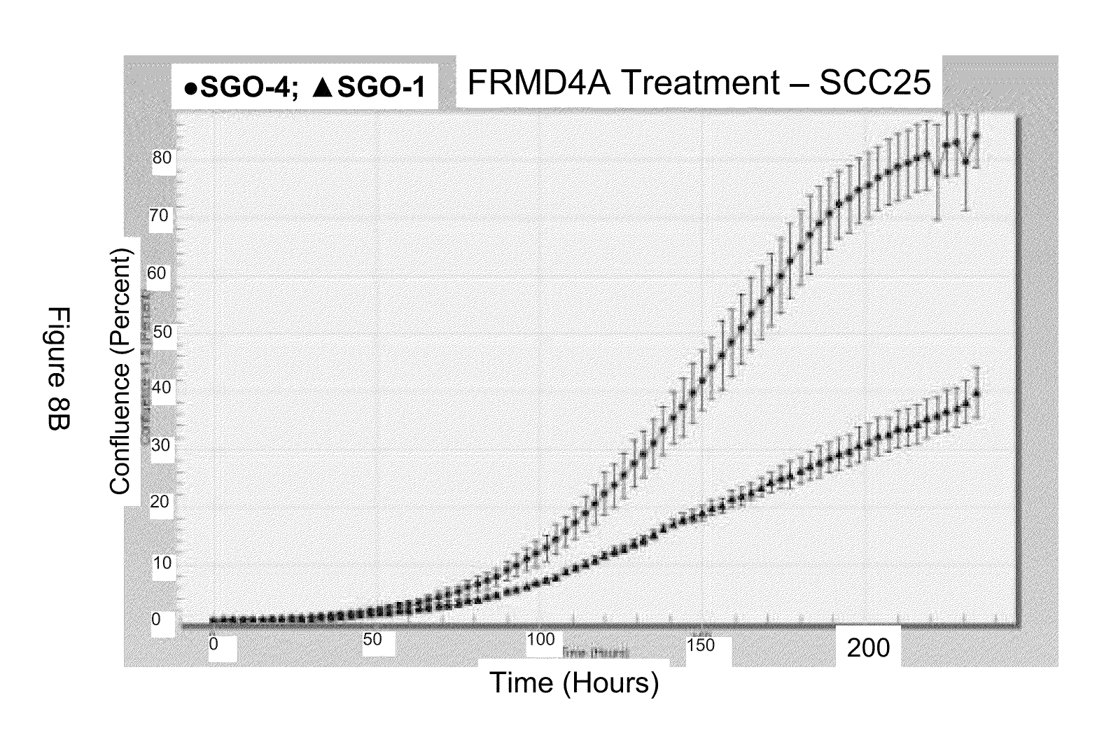

FRMD4A-shRNA knock down tumours were noted to be more keratinised, suggesting that they were more likely to contain differentiating cells. This was confirmed by co-staining xenograft tumour sections for FRMD4A and the differentiation marker involucrin. Empty vector (EV) control tumours stained positively throughout for FRMD4A but were negative for involucrin, while FRMD4A knockdown tumours showed the expected loss of FRMD4A staining, but with areas of strong involucrin staining (FIG. 7A).

### FRMD4A Influences Growth of SCCs by Modulating the Hippo Pathway

The Hippo pathway in mammals has been shown to regulate organ size in development and regrowth following injury. Contact inhibition of cells ensures that epithelial cells stop growing when the organ reaches the appropriate size. This ability to self regulate based on contact inhibition appears lost in cancers. Normal keratinocytes differentiate when suspended in methylcellulose. SCC13-WT and SCC13-SCR control cells are able to grow and form spheres when suspended at low density in methylcellulose, whereas SCC13-A7 has a much lower sphere forming ability; even less than its colony forming efficiency.

The final mediator of the Hippo pathway is the transcriptional co-activator YAP. Phosphorylation of YAP by LATS1/2 maintains YAP within the cytoplasm, whereas removal of Lats1/2 allows the unphosphorylated YAP to enter the nucleus where it is thought to play a role in transcriptional control of proliferation, apoptosis and differentiation. To test the whether FRMD4A plays a role in the Hippo pathway, SCC13-WT, —SCC13-SCR and SCC13-A7 cells were grown on coverslips and stained for YAP, LATS and MST. SCC13-WT and SCC13-SCR control cells showed generalised cytoplasmic staining with minimal nuclear accumulation. Upon knocking down FRMD4A a clear change in the staining pattern demonstrated a shift of YAP to the nucleus (FIG. 7B). Staining of LATS greatly reduced in the FRMD4A knockdowns, but little change was seen in the immunofluorescence for MST (supplemental data). Western blot analysis of SCC13-WT, SCC13-SCR and SCC13-A7 lysates confirmed an overall increase in levels of YAP on knocking down FRMD4A (FIG. 7B). Histological sections of xenografted SCC13 cells showed this pattern was also present in vivo (FIG. 7C).

### Modulating the Hippo Pathway to Inhibit the Growth of SCCs

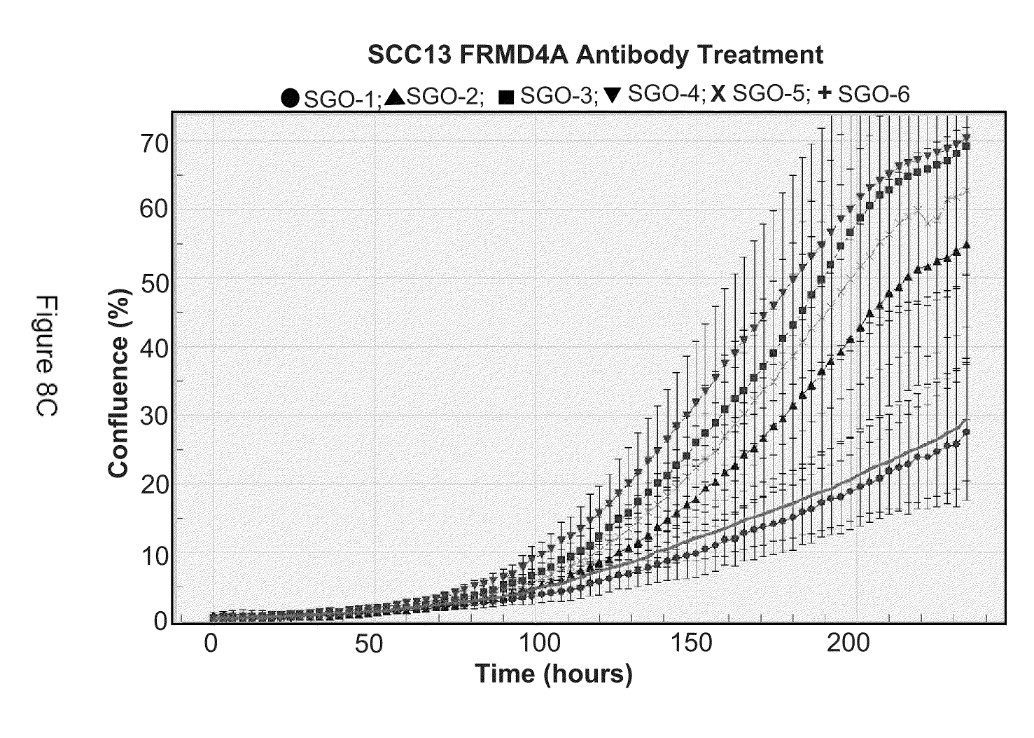

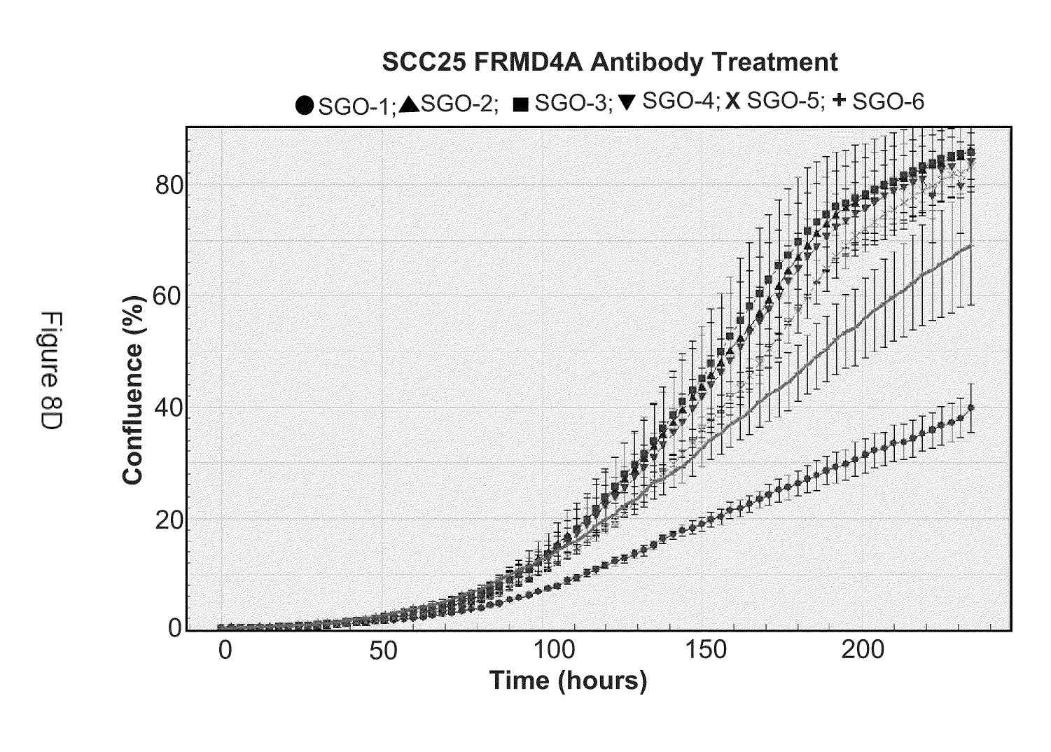

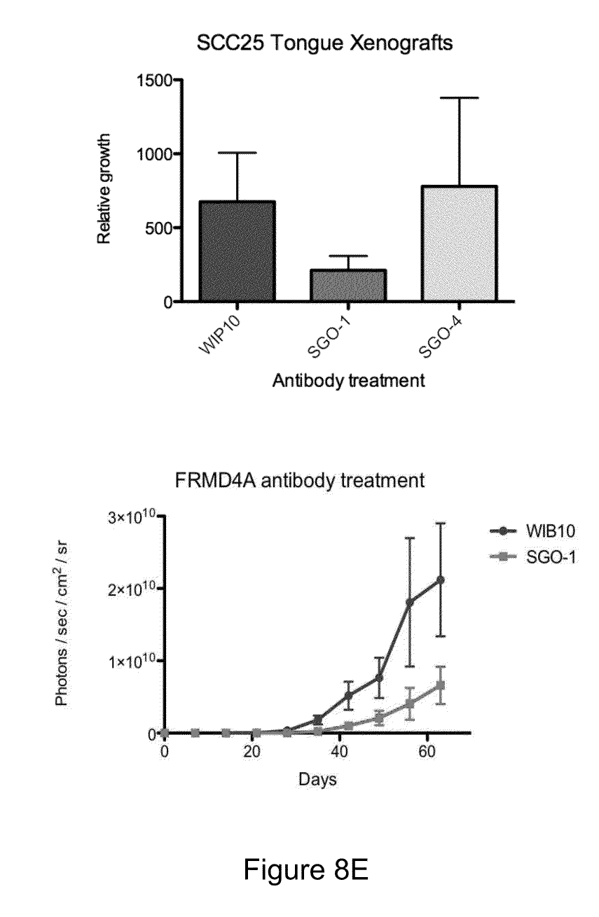

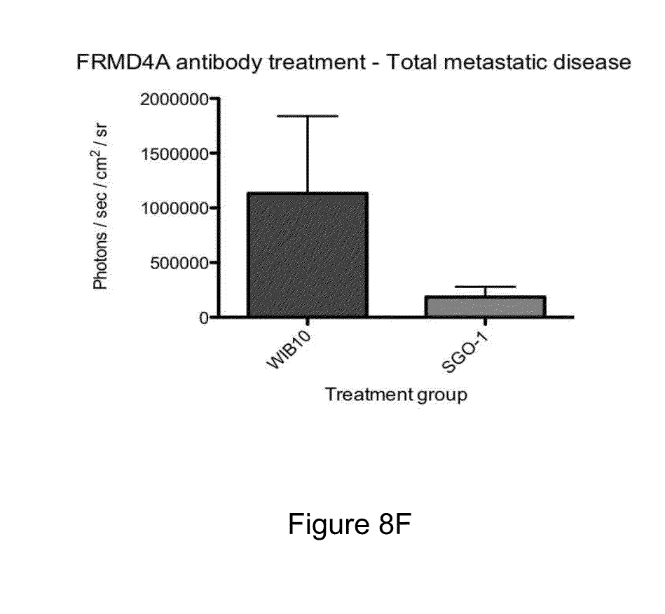

Having established that FRMD4A has a functional role in SCC proliferation and tumour growth and that it is also modulating the differentiation of such cells, in part perhaps via cancer stem cells and is a key mediator of the Hippo pathway control in SCCs, the present inventors recognised that FRMD4A represents a potential target for therapeutic options. Currently many targeted antibody therapies are in clinical use or undergoing trials to determine their efficacy in the treatment of several malignant and non-malignant diseases. To test if any of the generated antibodies developed against FRMD4A may have a blocking effect on SCCs, the antibodies were added to cells grown in the Incucyte timelapse imaging system. Of the six antibodies tested, one was found to be particularly effective in reducing the growth rate of SCC cells; that antibody was SGO-1 (see FIGS. 8A and 8B). SGO-6 also exhibited a reduction in SCC growth rate. SGO-2 and SGO-5 also exhibited a reduction in SCC growth rate, albeit that the magnitude of the effect was less pronounced in comparison with SGO-1 (see FIGS. 8C and 8D). Treatment with SGO-3 and SGO-4 did not exhibit any significant reduction in SCC cell growth rate under the conditions tested herein.

SGO-1 treatment showed an approximate 50% reduction in growth of cells over the time of the experiment, and the effect was consistent across the four different SCC cell lines used. The antibody SGO-1 was then tested in the tongue xenograft model, and again a single intraperitoneal dose of this anti-FRMD4A antibody slowed the growth of SCC25 tumours compared to mice treated with one control FRMD4A antibody and mice treated with an IgG control antibody WIB10 (FIG. 8E). Mice with SCC25 xenografts treated with the FRMD4A antibody SGO-1 also showed a significant reduction in metastatic disease burden as compared to the WIB10 control IgG antibody (FIG. 8F).

### FRMD4A Expression and Staining Across a Panel of Tumour Types:

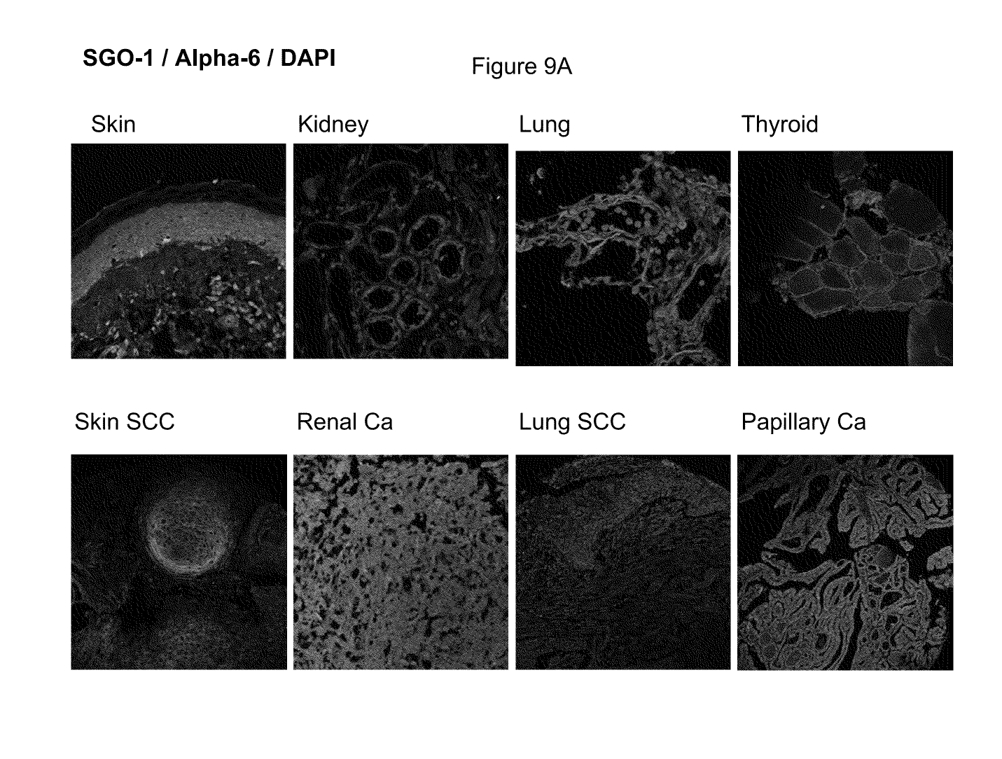

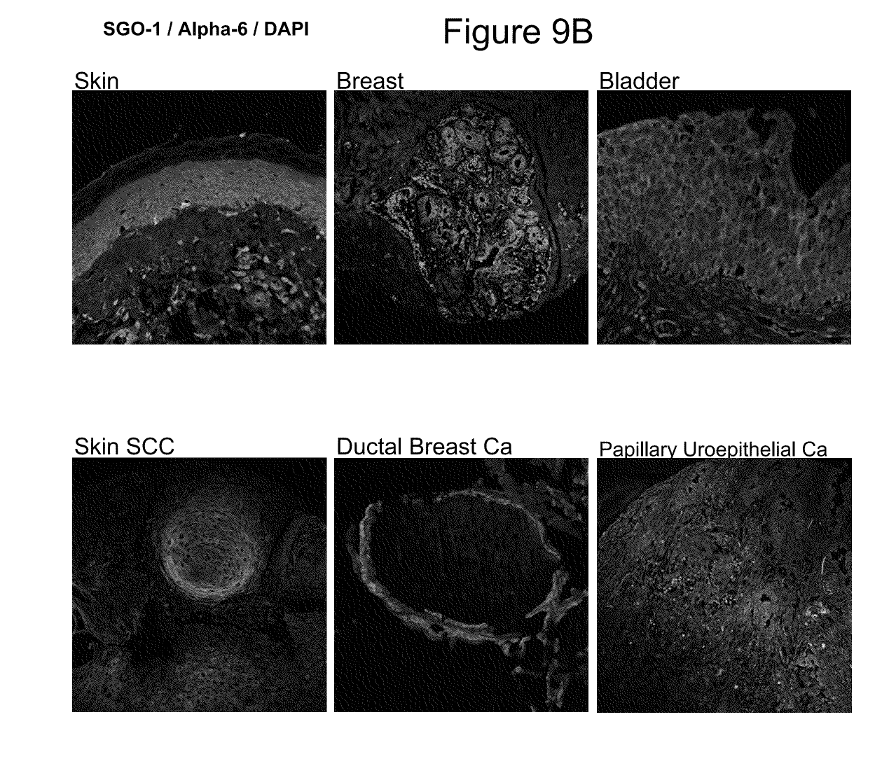

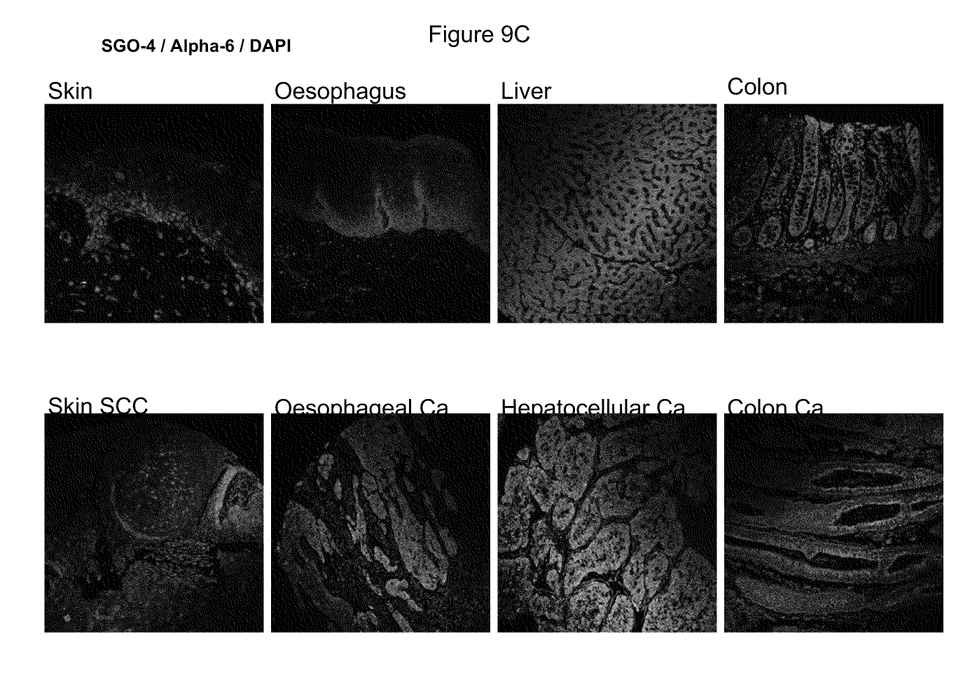

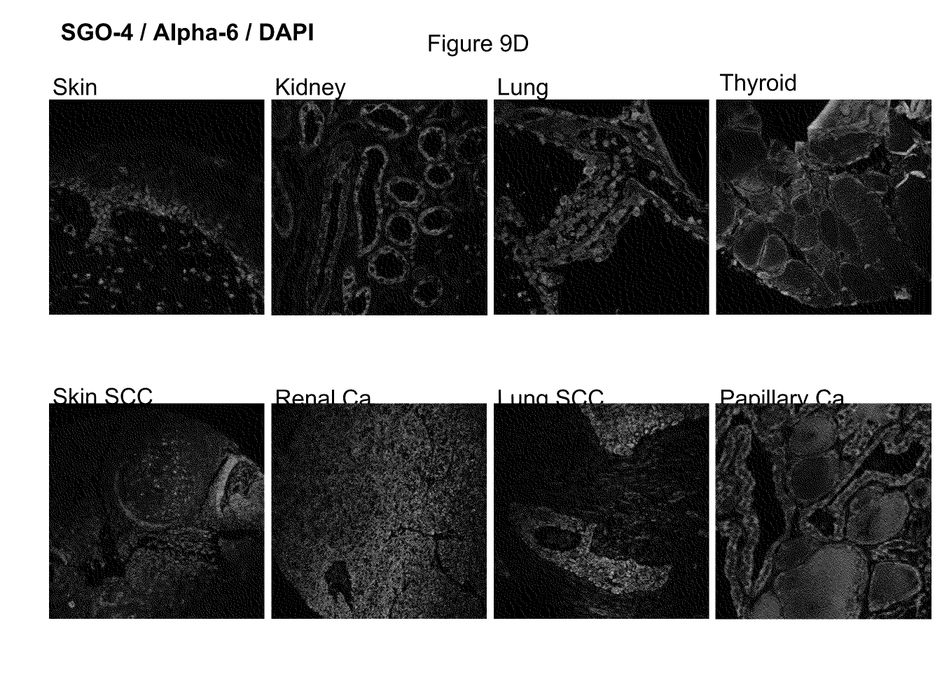

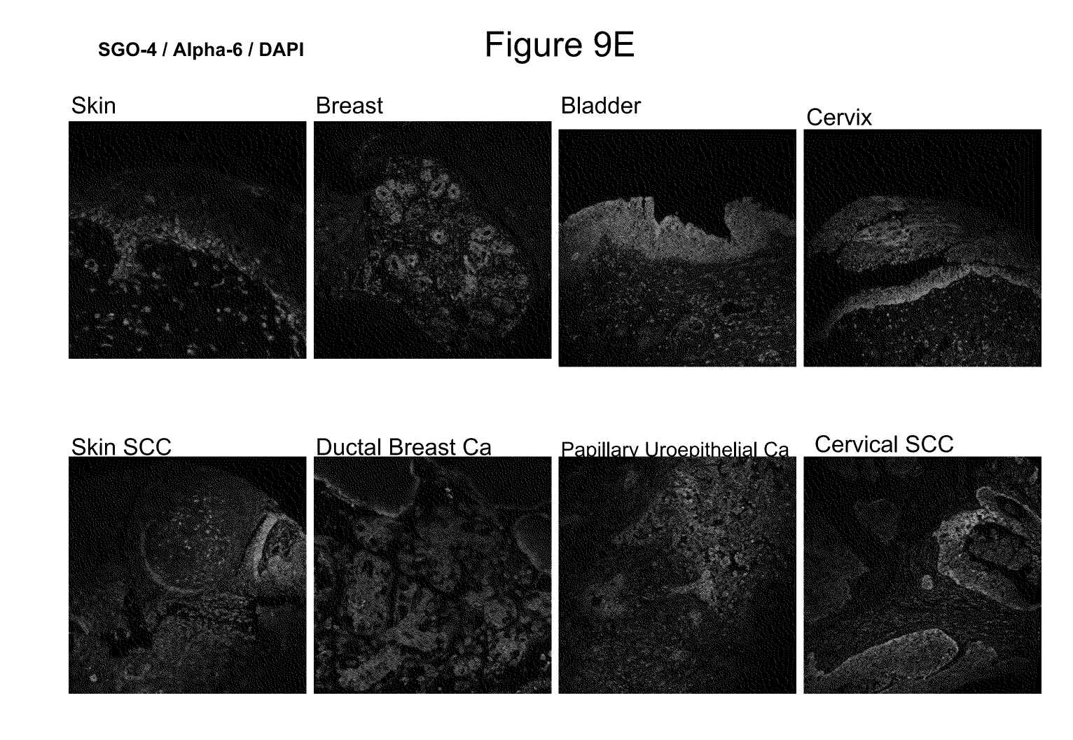

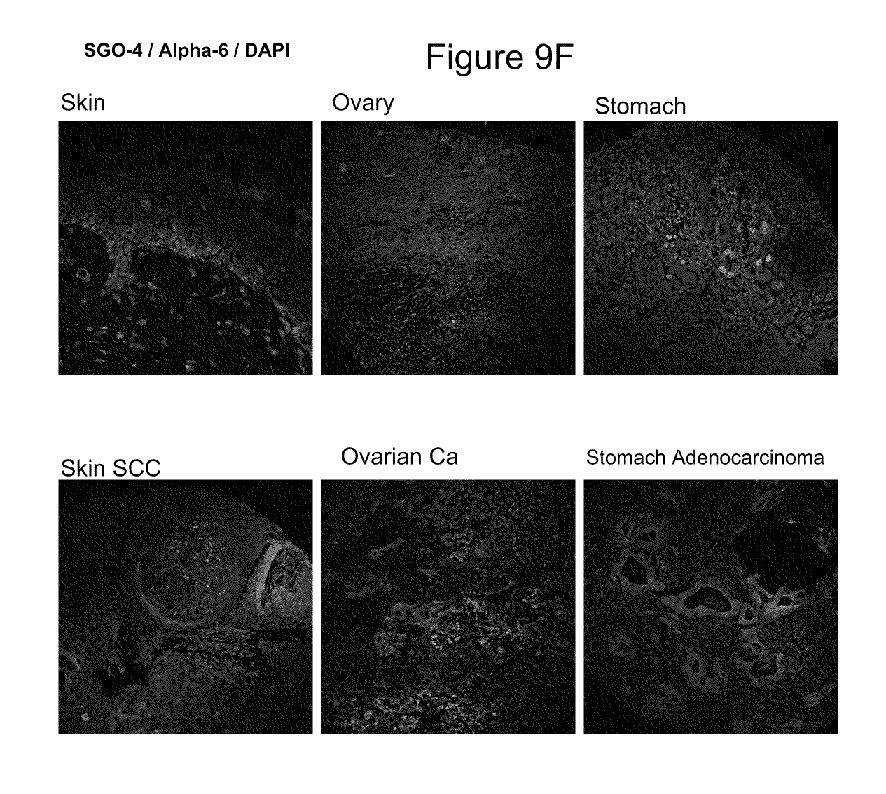

A tissue microarray containing human tumour tissue alongside matched samples of normal tissue was stained with antibodies against FRMD4A (FIG. 9). While in many normal tissues such as skin the staining of FRMD4A was localised to particular regions such as the basal layer (FIG. 9A to 9F), FRMD4A in tumour samples was found to be much more widespread in its expression pattern across each tumour tissue section (FIG. 9A-9F). As well as expression of FRMD4A being widespread in squamous cell carcinomas it was also detected in other tumours of epithelial origins such as carcinomas and adenocarcinomas. These results suggest that FRMD4A expression may be a more widespread phenomenon than simply SCC and that therapeutic antibodies to FRMD4A may therefore have utility in a much broader range of cancer types.

The cell surface hyaluronic acid (HA) receptor. CD44 is upregulated in many human cancers. The cytoplasmic domain of CD44 interacts with ERM proteins and Merlin and may play a role in activating the Hippo pathway in cancer (Xu et al., 2010). SCC13 cells grown on plates coated with HA showed a reduction in colony forming efficiency, reduction in average colony area and average colony staining intensity. Overall growth rate was measured using the Incucyte timelapse system, which showed a decrease in growth rate when SCCs were cultured on HA coated plates. Cells grown on HA coated coverslips showed an increase in CD44 staining, and a loss of nuclear FRMD4A staining. To test if HA may have a potential role as a therapeutic agent in vivo, mice were xenografted with SCC13 into their back skin. Tumours were injected weekly with HA or PBS and measured using the Xenogen IVIS. HA injected tumours showed a reduced growth rate compared with PBS injected tumours, suggesting this may be a therapeutic option, e.g. for SCCs which are not readily resectable by surgery.

Recent preclinical and clinical trials of HSP90 inhibitors have shown varying success in the treatment of several cancer types. Their effect could be mediated by depletion of LATS1/2 in the Hippo pathway (Huntoon et al., 2010). Depletion of LATS1/2 in this study reduced phosphorylation of YAP with resulting translocation to the nucleus. We tested HSP90 inhibitor, 17-DMAG, in our system to see if it had an effect on SCCs. SCC13 cells grown in the presence of 17-DMAG in vitro were readily killed over a range of concentrations. Mice were tongue xenografted and then given weekly i.p. injections of 17-DMAG or vehicle control. The mice in the 17-DMAG treated arm showed a slower progression of their tumours, compared to the control group. It is contemplated that an increased dosing regime may increase the effect seen on the xenografts.

### Discussion

Stem cells located in the interfollicular epidermis or the mucosa of the oral cavity divide to produce daughter cells, which commit to terminal differentiation. They gradually migrate through the levels of the epithelium before being shed. Genetic instability caused by environmental exposure or viral infection may distort this homeostatic process and induce a malignant transformation. In SCC the stem cell compartment expands, cells fail to downregulate integrin expression and they lose the drive to differentiate. Keratinocytes no longer respond to the cell-cell or cell-basement membrane communication and continue to grow and invade surrounding structures, before metastasising to distant organs. FRMD4A has been shown to be a marker of the cells populating the basement layer of the epidermis, and lost during differentiation. In specimens of human SCCs collected from the oral cavity of patients, FRMD4A expression is increased throughout the tumour.

The present inventors have found that knocking down expression of FRMD4A in human SCC lines decreases their ability to grow and invade in vitro. In vivo xenograft studies of the effect of FRMD4A knockdown also shows reduced growth of the primary tumour, along with a decreased ability to metastasise to the lungs or liver. Xenografted tumours with FRMD4A knock down also showed evidence of reduced ability to proliferate, they had an increased tendency to be apoptotic and also cells re-acquired the drive to differentiate.

The Hippo pathway has been implicated in the homeostatic control of organ size and of interest in cancer because of apparent loss of these regulatory mechanisms. FRMD4A shares some structural characteristics with upstream regulators of the pathway, such as Merlin and Expanded, which both contain a FERM domain. Reduced expression of FRMD4A using shRNA influenced downstream mediators such as LATS and YAP, allowing the latter to translocate to the nucleus and exert its transcriptional effects on proliferation, apoptosis and differentiation. Keratinocytes in suspension differentiate rapidly, due to their loss of cell-cell and cell-basement membrane interaction. Many cancer cell lines, including SCCs are able to grow in suspension due to a loss of contact inhibition. “The loss of contact inhibition and the gain of anchorage-independent growth are hallmarks of cancer cells in vitro” (Hanahan and Weinberg, 2000). Knockdown of FRMD4A in SCCs renders the cells no longer impervious to the loss of cell-cell contact and they fail to form spheres in methylcellulose. Loss of FRMD4A in tail-vein injected cells reduced the number of cells metastasising to the lungs, suggesting that these cells maybe be less able to survive anoikis and metastasise.

Having identified a cancer stem marker with an influential effect on a potential cancer pathway, the present inventors recognised that it would be attractive to modulate that target therapeutically. The experimental work described herein demonstrates that an antibody to FRMD4A may have an inhibitory functional role, as both in vitro and in vivo experiments using an anti-FRMD4A antibody therapeutically have shown a reduction in SCC cell and tumour growth.

CD44 is overexpressed in several cancers, including SCC of the head and neck. The hyaluronic acid receptor has been shown to be a potential marker of cancer stem cells in HNSCC (Prince et al., 2007) and it has an attenuating effect on the Hippo pathway in glioblastoma (Xu et al., 2010). The interaction between HA and CD44 was shown to reduce the metastatic potential of breast cancer cell lines (Lopez et al., 2005). In this study we show that treating cells in vitro with HA reduces the growth of SCCs, but also changes the nature of colonies formed to those associated with a differentiated cell status. Other studies have shown that smaller, more differentiated colonies are less likely to form xenograft tumours than larger colonies containing more “stem-like” cells (data not shown). Treatment of xenograft tumours with HA injection reduced the growth of the tumour and increased survival. This therapeutic option may be particularly attractive for patients where further management by surgery or adjuvant therapies is not possible, or concurrently with current treatment modalities. HA is currently used by injection in the skin for cosmetic treatments, and more recently into the knee joint for treatment of osteoarthritis, with no apparent toxic effects.

We have shown that an inhibitor of HSP90 drugs can interfere with the Hippo pathway in human SCC. 17-DMAG had a dramatic effect on in vitro experiments and a significant effect when used in vivo at a low dose. Phase I trials have determined a safe maximum dose for use of this drug in patients. The present inventors contemplate studying the efficacy of HA in the management of SCCs, including in man.

The role of the immune system in the pathogenesis and progression of malignant disease is an increasing focus for those studying cancer. The in vitro experiments and in vivo experiments described herein are such that the effects of the immune system have been deliberately obliterated as part of their design. However, the Hippo pathway itself appears to exert its role without regard to the immune system.

The subpopulation of cancer stem cells within a tumour has until recently been predicted to be relative small. Xenografts of single melanoma cells have been reported to form tumours in NSG mice with a frequency of approximately, one in four, which is much higher than had been suggested previously (Quitana et al., 2008). As in our experiments, the melanoma studys also used the NSG mouse as a host for xenografts. The authors state the further reduction in the immune system is responsible for the increased tumorigenicity in the NSG mouse compared to standard NOD/SCIDs. They also found that the addition of Matrigel made the xenografts grow faster, as did we in pilot experiments, however, it did not increase the percentage of mice that formed tumours [data not shown]. We chose not to add Matrigel or other support media/cells to our final model in order to reduce the effects of multiple variables. Our dilutional xenografts revealed that tongue tumours could be formed consistently with a minimum of 100 cells, whereas, back skin chamber grafts required 1×10E5 cells to establish a tumour. Knockdown of FMRD4A increased the number of cells required in order to establish a tongue tumour. Without wishing to be bound by any theory, the present inventors contemplate that knockdown of FMRD4A may reduce the number of cancer stem cells within the population.

SCCs developed faster when tongue grafted as opposed to back skin chamber grafts, regardless of the origin of the SCC from which the cells were derived. This suggest that all SCCs have the potential to grow aggressively, however, it is their anatomical location that dictates their natural history, rather than just the cytological or genetic variations of their tissue of origin. The tongue is essentially muscle coated in mucosa and therefore highly vascular. Without wishing to be bound by any theory, the present inventors contemplate that the increased vascularity in the tongue compared to the subcutaneous fascia in the back skin may be one factor that causes the differing grafting rates. More recent melanoma studies have suggested that cancer stem cells in melanoma are not truly as common as 27% and that foibles in the methodology led to an incorrect interpretation of results (Boiko et al., 2010). These authors found that serially passaging melanomas in vivo or in vitro in order to increase the number of cells available from a patient sample, may be allowing selection for more tumorigenic or stem cell-like cells. Therefore, in order to achieve accurate estimates of the percentage of the subpopulation of cancer stem cells, xenografts should be made directly from single cell suspension of the digested primary tumour. Our studies relied mainly on pre-existing cell lines, but did not detect obvious differences when compared to low passage cells derived directly from patient samples.

Human SCC in skin and the mucosa of the oral cavity remain a considerable cause of morbidity and mortality despite relative progress in the treatment of other epithelial cancers. FRMD4A and its role in the Hippo pathway may represent a potential opportunity to modify the disease and reduce its burden on patients.

### Example 2

### Monoclonal Anti-FRMD4A Antibodies

Monoclonal antibodies were raised against a peptide derived from human FRMD4A (DRRVLEHDFPKKSGPVVLYFC) SEQ ID NO: 6, which is residues 78 to 98 of the sequence set forth in SEQ ID NO: 1, using the HuCAL technology (AbD Serotec).

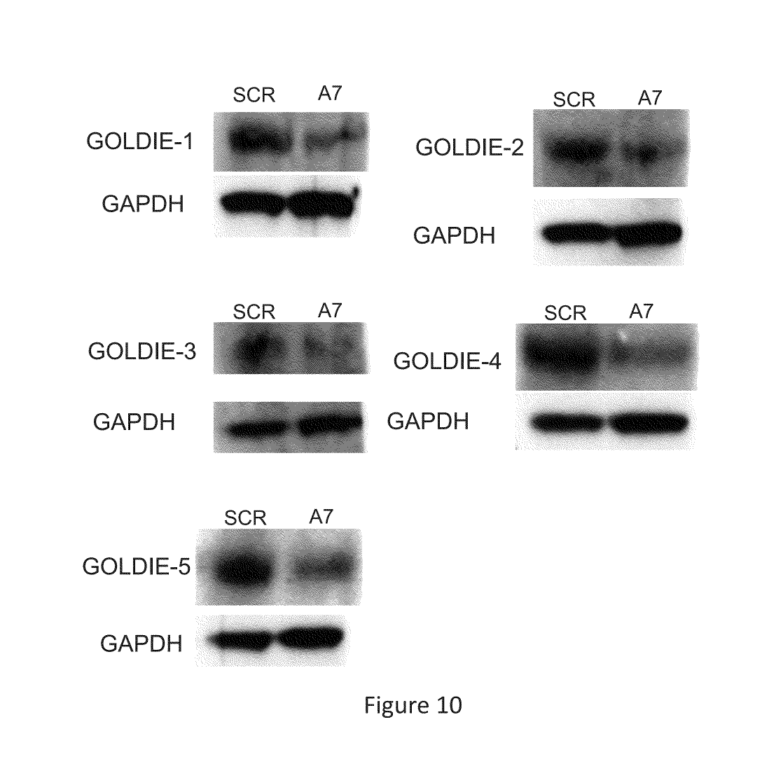

Western blot analysis was used to confirm monoclonal antibody binding to FRMD4A (see FIG. 10). SCC13 cells were treated with siRNA targeting human FRMD4A (A7) or scramble control siRNA (SCR) and levels of FRMD4A determined using 5 distinct monoclonal antibodies, (GOLDIE-1, GOLDIE-2, GOLDIE-3, GOLDIE-4 and GOLDIE-5). Loss of staining intensity was observed in the FRMD4A siRNA treated cells, demonstrating that the monoclonal antibodies bind to human FRMD4A. GAPDH was used to confirm equal protein loading.

### Example 3

### FRMD4A Antibody Efficacy Requires the Presence of FRMD4A

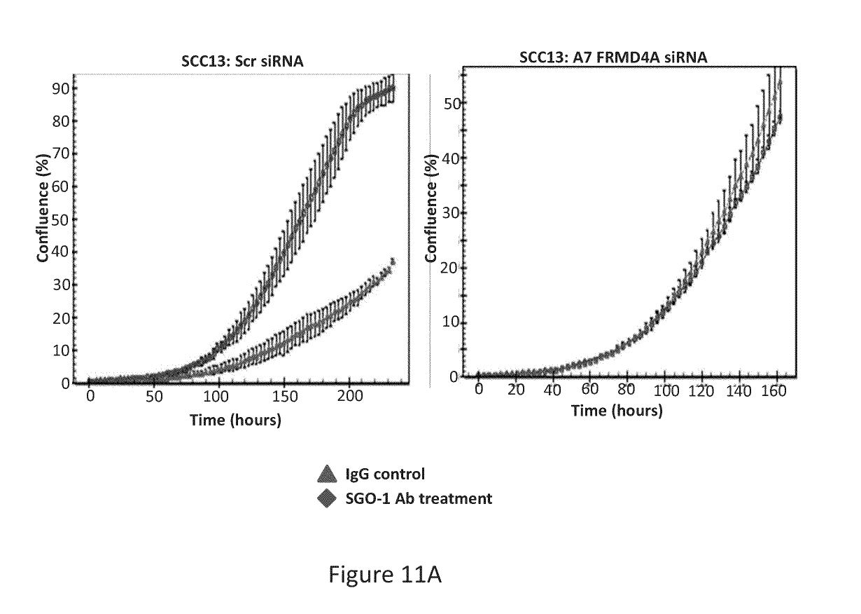

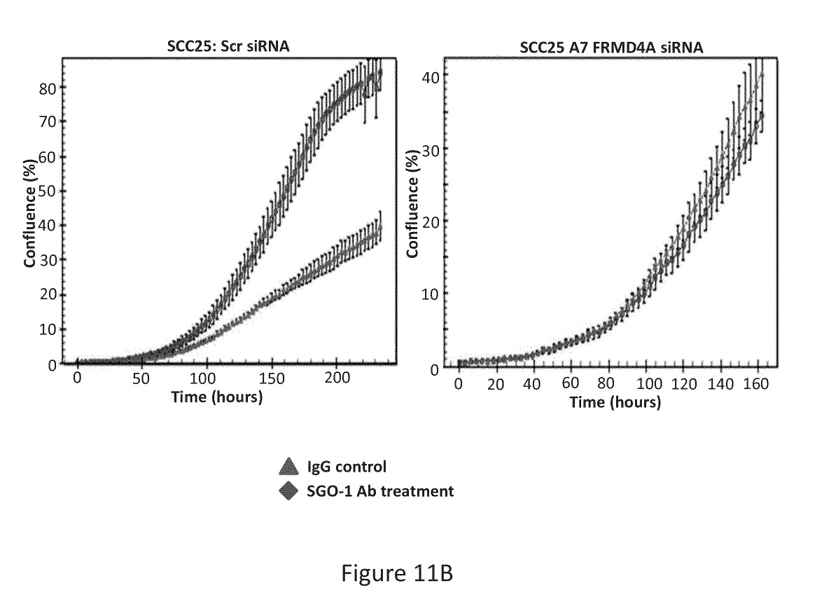

SCC25 and SCC13 cells were treated with either scramble control siRNA (Scr) or FRMD4A siRNA (A7) (see FIGS. 11A and 11B). Loss of FRMD4A protein expression was observed in the A7 treated cells. Treatment of the Scr cells with SGO-1 FRMD4A antibody reduced cell confluence. In contrast, in the absence of FRMD4A, A7 treated cells showed no reduction in confluence when treated with SGO-1 antibody. Therefore, antibody efficacy requires the presence of FRMD4A, suggesting that the antibody mediates its effects through binding to FRMD4A. This indicates that the anti-cancer activity of the FRMD4A antibody is the result of the antibody binding to FRMD4A.

Sequences

NCBI Accession No. NP_060497; GI: 116063562 (Human FRMD4A protein sequence)
SEQ ID NO: 1:

| | |
|---|---|
| 1 | mavqlvpdsa lgllmmtegr rcqvhllddr klellvqpkl lakelldlva shfnlkekey |
| 61 | fgiaftdetg hlnwlqldrr vlehdfpkks gpvvlyfcvr fyiesisylk dnatielffl |
| 121 | naksciykel idvdsevvfe lasyilqeak gdfssnevvr sdlkklpalp tqalkehpsl |
| 181 | aycedrvieh ykklngqtrg qaivnymsiv eslptygvhy yavkdkqgip wwlglsykgi |
| 241 | fqydyhdkvk prkifqwrql enlyfrekkf svevhdprra svtrrtfghs giavhtwyac |
| 301 | paliksiwam aisqhqfyld rkqskskiha arslseiaid ltetgtlkts klanmgskgk |
| 361 | iisgssgsll ssgsqesdss qsakkdmlaa lksrqealee tlrqrleelk klclreaelt |
| 421 | gklpveypld pgeeppivrr rigtafklde qkilpkgeea elerlerefa igsqiteaar |
| 481 | rlasdpnvsk klkkqrktsy lnalkklqei enainenrik sgkkptqras liiddgnias |
| 541 | edsslsdalv lededsqvts tisplhsphk glpprppshn rppppqsleg lrqmhyhrnd |
| 601 | ydkspikpkm wsessldepy ekvkkrsshs hssshkrfps tgscaeaggg snslqnspir |
| 661 | glphwnsqss mpstpdlrvr sphyvhstrs vdisptrlhs lalhfrhrss slesqgkllg |
| 721 | sendtgspdf ytprtrssng sdpmddcssc tshsssehyy paqmnanyst laedspskar |
| 781 | qrqrqrqraa galgsassgs mpnlaargga ggaggagggv ylhsqsqpss qyrikeyply |
| 841 | ieggatpvvv rslesdqegh ysvkagfkts nsytagglfk eswrggggde gdtgrltpsr |
| 901 | sqilrtpslg regandkgag raaysdelrq wyqrstashk ehsrlshtss tssdsgsqys |
| 961 | tssqstfvah srvtrmpqmc katsaalpqs qrsstpssei gatppssphh iltwqtgeat |
| 1021 | enspildgse spphqstde |

NCBI Accession No. NM_018027; GI: 116063561 (Human FRMD4A mRNA sequence).
SEQ ID NO: 2:

| | |
|---|---|
| 1 | gcagtgcaat tccatgttcc tcttaagtat gttagcccta ccgggagctg agctggccag |
| 61 | tctacttgga gaggaaaagt agatctgggg aaggtggaag ggtcagttcc taagtgactt |
| 121 | cctcctcggg gatggtaagg gcatttgctg atctccagtg actgcctggt gcctcatggt |
| 181 | cagactcggc tgtctcactc ccagatatct gattttgcaa aaagggacac acctatctgc |
| 241 | agcaaagaag acactgacca gattgcgagc ggtgcttttg gatgctctgt agccacccgg |
| 301 | ggcccaggag gactgactcg gcagcaggat tcgtgcatgg gaatcggaga ccatggcagt |
| 361 | gcagctggtg cccgactcag ctctcggcct gctgatgatg acggagggcc gccgatgtca |
| 421 | agtacatctt cttgatgaca ggaagctgga actcctagta cagcccaagc tgttggccaa |
| 481 | ggagcttctt gaccttgtgg cttctcactt caatctgaag gaaaaggagt actttggaat |
| 541 | agcattcaca gatgaaacgg gacacttaaa ctggcttcag ctagatcgaa gagtattgga |
| 601 | acatgacttc cctaaaaagt caggacccgt ggttttatac ttttgtgtca ggttctatat |
| 661 | agaaagcatt tcatacctga aggataatgc taccattgag cttttctttc tgaacgcgaa |
| 721 | gtcctgcatc tacaaggagc ttattgacgt tgacagcgaa gtggtgtttg aattagcttc |
| 781 | ctatatttta caggaggcaa agggagattt ttctagcaat gaagttgtga ggagtgactt |
| 841 | gaagaagctg ccagcccttc ccacccaagc cctgaaggag cacccttccc tggcctactg |
| 901 | tgaagacaga gtcattgagc actacaagaa actgaacggt cagacaagag gtcaagcaat |
| 961 | cgtaaactac atgagcatcg tggagtctct cccaacctac ggggttcact attatgcagt |
| 1021 | gaaggacaag cagggcatac catggtggct gggcctgagc tacaaaggga tcttccagta |
| 1081 | tgactaccat gataaagtga agccaagaaa gatattccaa tggagacagt tggaaaacct |
| 1141 | gtacttcaga gaaaagaagt tttccgtgga agttcatgac ccacgcaggg cttcagtgac |
| 1201 | aaggaggacg tttgggcaca gcggcattgc agtgcacacg tggtatgcat gtccggcatt |
| 1261 | gatcaagtcc atctgggcta tggccataag ccaacaccag ttctatctgg acagaaagca |
| 1321 | gagtaagtcc aaaatccatg cagcacgcag cctgagtgag atcgccatcg acctgaccga |
| 1381 | gacggggacg ctgaagacct cgaagctggc caacatgggt agcaagggga agatcatcag |
| 1441 | cggcagcagc ggcagcctgc tgtcttcagg ttctcaggaa tcagatagct cgcagtcggc |
| 1501 | caagaaggac atgctggctg ccttgaagtc caggcaggaa gctctggagg aaaccctgcg |
| 1561 | tcagaggctg gaggaactga agaagctgtg tctccgagaa gctgagctca cgggcaagct |
| 1621 | gccagtagaa tatcccctgg atccagggga ggaaccaccc attgttcgga gaagaatagg |
| 1681 | aacagccttc aaactggatg aacagaaaat cctgcccaaa ggagaggaag ctgagctgga |
| 1741 | acgcctggaa cgagagtttg ccattcagtc ccagattacg gaggccgccc gccgcctagc |
| 1801 | cagtgacccc aacgtcagca aaaaactgaa gaaacaaagg aaaacctcgt atctgaatgc |
| 1861 | actgaagaaa ctgcaggaga ttgaaaatgc aatcaatgag aaccgcatca agtctgggaa |
| 1921 | gaaacccacc cagagggctt cgctgatcat agacgatgga aacattgcca gtgaagacag |
| 1981 | ctccctctca gatgcccttg ttcttgagga tgaagactct caggttacca gcacaatatc |
| 2041 | ccccctacat tctcctcaca agggactccc tcctcggcca ccgtcgcaca acaggcctcc |
| 2101 | tcctccccag tccctggagg gactccgaca gatgcactat caccgcaacg actatgacaa |
| 2161 | gtcacccatc aagcccaaaa tgtggagtga gtcctcttta gatgaaccct atgagaaggt |
| 2221 | caagaagcgc tcctctcaca gccattccag cagccacaag cgcttcccca gcacaggaag |
| 2281 | ctgtgcggaa gccggcggag gaagcaactc cttgcagaac agccccatcc gcggcctccc |
| 2341 | gcactggaac tcccagtcca gcatgccgtc cacgccagac ctgcgggtcc ggagtcccca |
| 2401 | ctacgtccat tccacgaggt cggtggacat cagccccacc cgactgcaca gcctcgcact |
| 2461 | gcactttagg caccggagct ccagcctgga gtcccagggc aagctcctgg gctcggaaaa |
| 2521 | cgacaccggg agccccgact tctacacccc gcggactcgt agcagcaacg gctcagaccc |
| 2581 | catggacgac tgctcgtcgt gcaccagcca ctcgagctcg gagcactact acccggcgca |
| 2641 | gatgaacgcc aactactcca cgctqgccga ggactcgccg tccaaggcgc gccagaggca |
| 2701 | gaggcagcgg cagcgggcgq cgggcgcact gggctcagcc agctcgggca gcatgcccaa |
| 2761 | cctggcggcg cgcqggggtg cggggggcgc ggggggcgcg gggggcggtg tgtacctgca |
| 2821 | cagccagagc cagcccagct cgcagtaccg catcaaggag tacccgctgt acatcgaggg |
| 2881 | cggcgccacg cccgtggtgg tgcgcagcct ggagagcgac caggagggcc actacagcgt |
| 2941 | caaggctcag ttcaagacgt ccaactccta cacggcgggc ggcctgttca aggagagctg |
| 3001 | gcgcggcggc ggcggcgacg agggcgacac gggccgcctg acgccgtcgc gatcgcagat |
| 3061 | cctgcggact ccgtcgctgg gccgcgaggg cgcccacgac aagggcgcgg gccgtgccgc |
| 3121 | cgtctcagac gagctgcgcc agtggtacca gcgttccacc gcctcgcaca aggagcacag |
| 3181 | ccgcctgtcg cacaccagct ccacctcctc ggacagcggc tcgcagtaca gcacctcctc |
| 3241 | ccagagcacc ttcgtggcgc acagcagggt caccaggatg ccccagatgt gcaaggccac |
| 3301 | gtcagctgcc ttacctcaaa gccagagaag ctcgacaccg tcaagtgaaa ttggagccac |
| 3361 | ccccccaagc agcccccacc acatcctaac ctggcagact ggagaagcaa cagaaaactc |
| 3421 | acccattctg gatgggtctg agtctccacc tcaccaaagt actgatgaat agaggagcta |
| 3481 | caatgatagc tgtttcctgg attcctccct ctatccagaa ctagctgatg tccagtggta |
| 3541 | cgggcaggaa aaagccaagc ccgggaccct cgtgtgagcc agcccggcct aatctgaccg |
| 3601 | cctcaacgcc attctgagat cacctcactg cctctcattt gccttaccca gacgcaccgt |
| 3661 | caccctgcac cagctttggc cctcagcact ttttttctcc tgtctccgca ttccctcccc |
| 3721 | cttgaaaacc tgactgagga gacattctgg aaggttccgg tcccactgtg tgtcccctgg |
| 3781 | cgctcttgcc catagagagc cagacaccaa tcctcaatgg caccttggtg gcttccctct |
| 3841 | gccatgacag cccctaggcc aggaaccatc aggggggcca gccggcatcc aattcctgcg |
| 3901 | gataagtagc gttgggagag aacgggaaag gggacttgag ttacagggtg acccagaaag |
| 3961 | acgattcagc tgtgtccagc ctgccaccca tacgtaggcc aaccaagcac ttcatgaaga |
| 4021 | ggaggcctcg tggcatattc agtttacacc tgaaatattc cttgatggga cagcttgtgg |
| 4081 | ggatggctat gggggaaggg gaggttgaga aaggaagttc tcgacaccaq aaatgcatcg |
| 4141 | gaggaccaca atcagttcta tgctgccaaa gattaaaaat aaataaaaac ataaaaaatt |
| 4201 | aagaggggcc aagaggaaga cattctttct gcaaggaaat ttcttttaaa ttctgaactg |
| 4261 | ctactacaca caagtgaaag tcaaccctat gtaaactggt gtcctctctc tagccctctc |
| 4321 | ccttactggc ccacttctct ctccgtagag agcctgaaaa actgccccaa tgccacggta |
| 4381 | aaggcgagga agtcttggct ggcgttgctg actcacagtc gccatccatc tggacacaaa |
| 4441 | gagagacctg tgggagtcgt agagggtact gttagccccg gtccatgcag ggggttcagc |
| 4501 | cgagcccaag actcaaagct gctttccttt caggatttgt agtaacgtaa ggtgataatg |
| 4561 | gccaaaagtg gttctctctc attaaaccaa ccagtaaaag cgtatcctat ttttttgcac |
| 4621 | aaggtgtttc attttcgttt ttatgggaaa ccaagggaaa agcacattgc gatccattca |
| 4681 | gtgtttaact gtcgtggctc attttctgtt cgttagcact tgtgtgacaa aagagctcag |
| 4741 | atccgacttc tcctatgtgt cacttattcc aagaacccaa ctatgccctt aggtagaaag |
| 4801 | atttqactcg tgtgtctact agccaacagg cagagcaggg ttgaaaaaaa tatcagctcc |
| 4861 | caaagggccc atgtgtotac atcatcagtt actgtcatgc accacatttq tgtgcagata |
| 4921 | ccaaaagagg aggaaagaag aaaaaaatta atgtgtggga gctgcacgtt tacatgtttL |
| 4981 | gagctatgct tcaaacacaa ctggaaagcc atcaatcttc aaaggcctca aaaatacttt |
| 5041 | tatagtaaca agtgcacgac tttagttggg ttattcaaga tggcacaaaa aggtttccgc |
| 5101 | agaggtggta tgctgtgctt ttggcgcaag tggtgggggg atggggtggg ggtggaattt |
| 5161 | ttttctcact ctaatgactt cctattggaa aggcattgac agccagggac aggagccagg |
| 5221 | gtgggggtag ttttgtggga aagcagaact gaagttagct taagcataaa aacaaagaaa |
| 5281 | aatcttcgct tttcatgtat gtggaatcca agaataacca taggctctac cagaccagaa |
| 5341 | gggtaaggat ggacactaaa atgaaacaaa taccaaggta ttccttctgc tgcagcctgg |
| 5401 | agaccaccga gagtcgagct ggggcacaca cacacctggc cgggacccgg cagggacaag |
| 5461 | gcgggccgtg gcctcctcca ccaagtctct ctagacaatt cagggcctgc tttccccagc |
| 5521 | tccatgcatg gctggactgg tgattccagg gtgcagaagg gattcatatt cccagaacgc |
| 5581 | tttaagtgta cacctgcagg ataaagagat accggttaca ttattaaatg attctaggga |
| 5641 | ttcactgggg gatatttttg ttgcttttac tttcatggtt agagctacaa agaacagtga |
| 5701 | tttttttttt ttctcccttc cccattcaga aacattatac attgggccat ttttctttct |
| 5761 | cccaaagaag attcatggat agtcagactg aactgtgtgc aacaggaaaa gtcaaaaggg |
| 5821 | aaaaggtagc tgatgaggtt acatggttac atgttctaca tcatgcatag tagcttgaaa |
| 5881 | tctagtctgg agaaaactgg atcaagattc tagcccactg gagttgcaag gaatgagagg |
| 5941 | caaaaattct aaagatttgg gttatatttt caacttgggg gacagagaga aatggagagc |
| 6001 | aggaattaca gttccaacaa acatcatgat agtctggtag tcaagacaga gattaagtaa |
| 6061 | aacaggtttt actgtttagc tgagttcagt taatacaaaa tgtacataaa acgttagtcc |
| 6121 | tttgagactg acatgattaa tgatcagtgt ggtgggaaat gatgtagtta ttgtacacaa |
| 6181 | gcacttgcaa actctttatc cctatttctt taaaacaaaa taaggtgaaa tacgaagtcc |
| 6241 | ttggtctgat ataaagcccc tattggattc ttcggatgcg taaaagaaat tgcctgtttc |
| 6301 | agccagaaga ctggtgaaaa cacatacatc agactatgtt gtgagccagg ttgatttttt |
| 6361 | attttattat atgcaggtga gtgttgaaac tgttaaaatt ccaatttgtt ttcattcagt |
| 6421 | attagtttag ttctaaatat agcaaacccc atccaggtgc tatcagatga ccagttactg |
| 6481 | cttagttaac taggtgtaaa gttttacata tacattaatg tcaatagttt attacaagtt |
| 6541 | gtgtaaaatg gactctagtt taataatggg ggaaaaaaga ttaggttgct cctgaaactg |
| 6601 | actgtagagc atgtaaaatg attttactgg attctgttca actgtaatca atgaaaaaga |
| 6661 | tgtacgttgt agacaaagtt gcagaattaa aaaaagaaat ctgcttttaa tttattcttt |
| 6721 | ttgtattaag aatttgtata gtacctttac attttgcaaa acagtgttgt caacacttat |
| 6781 | taaagcattt tcaaaatgaa aaga |

Residues 63-83 of SEQ ID NO: 1:
SEQ ID NO: 3:
IAFTDETGHLNWLQLDRRVLE
Residues 568-588 of SEQ ID NO: 1:
SEQ ID NO: 4:
PHKGLPPRPPSHNRPPPPQSL
Residues 1019-1039 of SEQ ID NO: 1:
SEQ ID NO: 5:
ATENSPILDGSESPPHQSTDE
Residues 78-98 of SEQ ID NO: 1:
SEQ ID NO: 6:
DRRVLEHDFPKKSGPVVLYFC
Residues 20-322 of SEQ ID NO: 1:
SEQ ID NO: 7:
RRCQVHLLDDRKLELLVQPKLLAKELLDLVASHFNLKEKEYFGIAFTDETGHLNWLQLDRRVLEHDFPKKS
GPVVLYFCVRFYIESISYLKDNATIELFFTLNAKSCIYKELIDVDSEVVFELASYILQEAKGDFSSNEVVR
SDLKKLPALPTQALKEHPSLAYCEDRVIEHYKKLNGQTRGQIAVNYMSIVESLPTYGVHYYAVKDKQGIPW
WLGLSYKGIFQYDYHDKVKPRKIFQWRQLENLYFREKKFSVEVHDPRRASVTRRTFGHSGIAVHTWYACPA
LIKSIWAMAISQHQFYLDRK

### EQUIVALENTS

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

All references, including patent documents, disclosed herein are incorporated by reference in their entirety for all purposes, particularly for the disclosure referenced herein.

### REFERENCES

Boiko, A. D. et al.
Human melanoma-initiating cells express neural crest nerve growth factor receptor CD271
Nature, 466, pp. 133-138
2010
Hanahan, D. and Weinberg, R. A.
The hallmarks of cancer
Cell, 100, pp. 57-70
2000
Huntoon, C. J. et al.
Heat shock protein 90 inhibition depletes LATS1 and LATS2, two regulators of the mammalian hippo tumor suppressor pathway
Cancer Res., 70, pp. 8642-8650
2010
Ikenouchi, J. and Umeda, M. FRMD4A regulates epithelial polarity by connecting Arf6 activation with the PAR complex
Proc. Natl. Acad. Sci. USA, 107(2), pp. 748-753
Jensen, K. B. and Watt, F. M.
Single-cell expression profiling of human epidermal stem and transit-amplifying cells: Lrig1 is a regulator of stem cell quiescence
PNAS, 103, pp 11958-11963
2006
Jensen, K. B. et al.
A stem cell gene expression profile of human squamous cell carcinomas
Cancer Letters, 272, pp. 23-31
2008
Locke, M. et al.
Retention of intrinsic stem cell hierarchies in carcinoma-derived cell lines
Cancer Res., 65, pp. 8944-8950
2005
Lopez, J. I. et al.
CD44 attenuates metastatic invasion during breast cancer progression
Cancer Res., 65, pp. 6755-6763
2005
Prince, M. E. et al.
Identification of a subpopulation of cells with cancer stem cell properties in head and neck squamous cell carcinoma
PNAS, 104, pp. 973-978
2007
Quintana, E. et al.
Efficient tumour formation by single human melanoma cells
Nature, 456, pp. 593-599
2008
Reya, T. et al.
Stem cells, cancer, and cancer stem cells
Nature, 414, pp. 105-111
2001
Xu, Y. et al.
CD44 attenuates activation of the hippo signaling pathway and is a prime therapeutic target for glioblastoma
Cancer Res., 70, pp. 2455-2464
2010

1. A method of treating a cancer in a mammalian subject, the method comprising administering a therapeutically effective amount of an antagonist antibody molecule that specifically binds to FERM domain-containing protein 4A (FRMD4A) to said subject, wherein the cancer is selected from: squamous cell carcinoma (SCC), an epithelial cancer, an adenocarcinoma and a carcinoma.

2. (canceled)

3. The method according to claim 1, wherein said FRMD4A has at least 90% amino acid sequence identity with the full-length of the amino acid sequence set forth in SEQ ID NO: 1.

4. The method according to claim 3, wherein said FRMD4A is human FRMD4A having the amino acid sequence set forth in SEQ ID NO: 1.

5. (canceled)

6. The method according to claim 1, wherein said antagonist antibody molecule causes at least 10% reduction in the growth rate, proliferation and/or cell number of cultured SCC cells in vitro as compared with cultured SCC cells grown under identical conditions, but in the absence of said antagonist antibody molecule.

7. The method according to claim 1, wherein said antagonist antibody molecule is selected from: a polyclonal antibody, a monoclonal antibody, an intrabody, a complete antibody, a single domain antibody, a nanobody, a Fab fragment, a F(ab′)2 fragment, a scFv, a diabody, a triabody, a human antibody, a humanised antibody, a bispecific antibody and a chimeric antibody.

8-14. (canceled)

15. The method according to claim 1, wherein said cancer is a cancer of a tissue or organ selected from: skin, oral cavity, tongue, head, neck, lips, mouth, oesophagus, urinary bladder, prostate, lung, vagina, cervix, kidney, thyroid, mammary papilla, breast, liver and colon.

16. The method according to claim 15, wherein the cancer is SCC of the skin, head and/or neck.

17. The method according to claim 1, wherein said method of treating the cancer comprises:
a decrease in the rate of growth;
an increase in apoptosis;
an increase in the cellular differentiation;
a decrease in metastasis; and/or
a decrease in the invasion,
of one or more cancer cells.

18. The method according to claim 1, wherein said method of treating the cancer comprises reducing the cell number and/or proliferation of at least one cancer stem cell.

19. An antagonist antibody molecule that specifically binds to FERM domain-containing protein 4A (FRMD4A) or a fragment thereof, wherein said antagonist antibody molecule binds to the region corresponding to or defined by residues 20-322 of the sequence of SEQ ID NO: 1, or the region corresponding to or defined by residues 1019-1039 of the sequence of SEQ ID NO: 1 (SEQ ID NO: 5).

20-31. (canceled)

32. A method of screening antibody molecules that specifically bind to FERM domain-containing protein 4A (FRMD4A), the method comprising:
(i) providing a plurality of antibodies directed to a FRMD4A polypeptide and/or one or more peptide fragments of said FRMD4A polypeptide; and
(ii) screening said antibodies from (i) for the ability to decrease the growth of SCC cells as compared with the growth of SCC cells not treated with said antibodies; and
optionally (iii) isolating the or those antibodies that screen positive for said ability in (ii); and
optionally (iv) determining the sequence of at least the complementarity determining regions (CDRs) of one or more of said antibodies isolated in (iii).

33. (canceled)

34. The method according to claim 32, wherein said antibody molecule is an antagonist antibody molecule that specifically binds to FERM domain-containing protein 4A (FRMD4A) or a fragment thereof, wherein said antagonist antibody molecule binds to the region corresponding to or defined by residues 20-322 of the sequence of SEQ ID NO: 1, or the region corresponding to or defined by residues 1019-1039 of the sequence of SEQ ID NO: 1 (SEQ ID NO: 5).

35-38. (canceled)

39. The method according to claim 1, wherein said antagonist antibody molecule binds to FRMD4A in the region corresponding to or defined by residues 20-322 of the sequence of SEQ ID NO: 1, or in the region corresponding to or defined by residues 1019-1039 of the sequence of SEQ ID NO: 1 (SEQ ID NO: 5).

40. The method according to claim 39, wherein said antagonist antibody molecule binds to FRMD4A in the region corresponding to or defined by residues 63-83 of the sequence of SEQ ID NO: 1 (SEQ ID NO: 3) or in the region corresponding to or defined by residues 78-98 of SEQ ID NO: 1 (SEQ ID NO: 6).

41. The method according to claim 1, wherein said antagonist antibody molecule is additionally conjugated or linked to a therapeutically active and/or cytotoxic moiety.

42. The method according to claim 19, wherein said antagonist antibody molecule binds to the region of FRMD4A corresponding to or defined by residues 63-83 of the sequence of SEQ ID NO: 1 (SEQ ID NO: 3) or the region corresponding to or defined by residues 78-98 of SEQ ID NO: 1 (SEQ ID NO: 6).

43. The method according to claim 19, wherein said antagonist antibody molecule causes at least 10% reduction in the growth rate, proliferation and/or cell number of cultured SCC cells in vitro as compared with cultured SCC cells grown under identical conditions, but in the absence of said antagonist antibody molecule.

44. The method according to claim 19, wherein said antagonist antibody molecule is selected from: a polyclonal antibody, a monoclonal antibody, an intrabody, a complete antibody, a single domain antibody, a nanobody, a Fab fragment, a F(ab′)2 fragment, a scFv, a diabody, a triabody, a human antibody, a humanised antibody, a bispecific antibody and a chimeric antibody.

45. The method according to claim 32, wherein said method of screening antibody molecules is an in vitro or an in vivo method of screening.
